(19) United States
(12) Patent Application Publication
(10) Pub. No.: US 2010/0223687 A1
(43) Pub. Date: Sep. 2, 2010

(54) COMPOSITIONS AND METHODS FOR DIAGNOSIS OF GENETIC SUSCEPTIBILITY, RESISTANCE, OR TOLERANCE TO INFECTION BY MYCOBACTERIA AND BOVINE PARATUBERCULOSIS

(75) Inventors: Holly L. Neibergs, Palouse, WA (US); Ricardo Zanella, Paim Filho (BR); Matthew Settles, Pullman, WA (US); Robert H. Whitlock, Kennett Square, PA (US); Jeremy F. Taylor, Columbia, MO (US)

Correspondence Address: DAVIS WRIGHT TREMAINE, LLP/Seattle, 1201 Third Avenue, Suite 2200, SEATTLE, WA, 98101-3045, US

(73) Assignee: Washington State University, Pullman, WA (US)

(21) Appl. No.: 12/620,524

(22) Filed: Nov. 17, 2009

Related U.S. Application Data
(60) Provisional application No. 61/199,550, filed on Nov. 17, 2008.

(51) Int. Cl. A01K 67/02; C12Q 1/68

(52) U.S. Cl. 800/22; 435/6

(57) ABSTRACT

Provided are methods for determining resistance or tolerance of a subject to Mycobacterium avium subspecies paratuberculosis (Map) infection, comprising determining, a presence or absence of a mutation, or the genotype of a single nucleotide polymorphism (SNP) within Map infection linkage disequilibrium region SEQ ID NO:46 that segregates with resistance or tolerance to Map infection, or determining a presence or absence of a mutation or a genotype of a SNP within a Map infection linkage disequilibrium region (SEQ ID NO:1) that is in linkage disequilibrium therewith, and determining, based thereon, susceptibility, resistance or tolerance of the subject to Map infection. In particular aspects, a presence or absence of a mutation, or the genotype of a single nucleotide polymorphism (SNP) within at least one gene selected from the group consisting of HIVEP3; EDN2; and LOC521287 that segregates with resistance or tolerance to Map tissue infection is determined.

### CROSS-REFERENCE TO RELATED APPLICATIONS

This application claims the benefit of priority to U.S. Provisional Patent Application Ser. No. 61/199,550, filed Nov. 17, 2008 and entitled GENETIC LOCI ASSOCIATED WITH JOHNE′S DISEASE, incorporated herein by reference in its entirety.

### STATEMENT REGARDING FEDERALLY-SPONSORED RESEARCH

This invention was made with government support under National Research Initiative grants 2005-35205-15448, 2005-35604-15615, 2006-35205-16701, and 2006-35616-16697 from the USDA Cooperative State Research, Education, and Extension Service. The Government has certain rights in the invention.

### TECHNICAL FIELD

The invention relates generally to genome-wide association studies to identify genetic loci associated with susceptibility, resistance or tolerance to disease, and in more particular aspects to identifying bovine genetic loci associated with susceptibility, resistance or tolerance to infection by Mycobacteria and Paratuberculosis, and to compositions and methods for diagnosis thereof. Certain aspects relate to methods comprising determination of a presence or absence of a mutation, or the genotype of a single nucleotide polymorphism (SNP) within at least one gene selected from the group consisting of HIVEP3; EDN2; and LOC521287 that segregates with resistance or tolerance to Map tissue infection.

### BACKGROUND

Bovine Paratuberculosis, commonly referred to as Johne's disease, is a contagious bacterial disease estimated to be present in 68.1% of U.S. dairy herds (APHIS 2008) and results in annual losses exceeding U.S. $200 million (Ott et al. 1999). The bacterium Mycobacterium avium subspecies paratuberculosis (Map) is responsible for Johne's disease and causes reduced milk production, reproductive failure, weight loss, and eventual death. Johne's disease is not treatable, and vaccination against Map has been largely unsuccessful.

The identification of animals with genetic susceptibility, resistance, or tolerance to infection by Map would provide mechanisms to reduce the incidence of Johne's disease. Further, a reduction of Map in the environment may also be beneficial to humans, as the presence of Map has also been implicated in the etiology of Crohn's disease (Bentley et al. 2008). It has been demonstrated that the genetic background of an animal plays a role in its resistance to Johne's disease (Settles et al, 2009). Cattle that are exposed to Mycobacterium avium subspecies paratuberculosis (Map) respond by either resisting or clearing the infection or becoming chronically infected with varying levels of disease severity.

Resistance or susceptibility to Map infection has been shown to have a hereditary component in cattle and mice (Koets et al. 2000; Mortensen et al. 2004; Gonda et al. 2006; Hinger et al. 2008), with estimates ranging from 0.06 to 0.102. However, attempts to locate genetic loci associated with resistance to paratuberculosis have had limited success. Gonda et al. (2007) found evidence for a quantitative trait locus (QTL) on Bos taurus chromosome 20 (BTA20) associated with paratuberculosis susceptibility. Hinger et al. (2007) investigated the association with paratuberculosis of 8 microsatellites located in or near Map susceptibility candidate genes in 1,179 (594 positive) German Holstein cows. However, none showed any significant associations. While Map susceptibility genes have yet to be identified in the bovine, Reddacliff et al. (2005) found an association of one microsatellite allele in SLC11A1 (formerly NRAMP1) with Map resistance in sheep.

Resistance to Map has been shown in mice to be associated with the Bcg gene or nramp1 which encodes the natural resistance-associated macrophage protein (Frelier et al. 1990; Skamene, 1989; Skamene et al. 1982). C57/B6 and BALB/c mice have the susceptible allele of Bcg and are susceptible to Map infections, while the C3H/HeJ strain is resistant to Map (Veazey et al. 1995a; Veazey et al. 1995b; Tanaka et al. 1994; Chiodini et al. 1993; Chandler 1962; Tanaka et al. 1994). In cattle, Map-susceptible Holstein sire lines have been found to be infected twice as often as resistant lines (Gonda et al. 2006). Heritability studies have been conducted on the presence or absence of disease based on postmortem tissue, ELISA and combined ELISA-fecal culture tests. In a Dutch study, the heritability of paratuberculosis infection was evaluated among vaccinated and unvaccinated animals based on findings from postmortem examinations (Koets et al. 2000). A heritability of 0.09, 0.01 and 0.06 was found for vaccinated, unvaccinated and all cows, respectively. A second study estimated the heritability of antibody response using a bivariate model with daily milk yield and optical density values from milk ELISAs (Mortensen et al. 2004). Mortensen and coworkers (2004) estimated the heritability to be 0.102 with the bivariate model and 0.091 when a sire model was used. Gonda and colleagues (2006) estimated the heritability of Johne's disease to be 0.153 based on fecal culture diagnostic testing, 0.159 based on ELISA and 0.102 from the combined antibody and fecal culture tests. We (Zanella et al. 2008) estimated the heritability of tolerance to Johne's disease to be 0.09.

Limited investigations have been conducted to identify loci associated with Johne's disease. Using a candidate gene approach, Taylor and colleagues (2006) evaluated the allele frequencies of a functional candidate gene, CARD15, in 30 unrelated unaffected animals and 11 affected animals without finding evidence for an association. Hinger et al. (2007) also investigated the association with Johne's disease utilizing 8 microsatellite genetic markers located in or near Map susceptibility candidate genes in 1,179 (594 positive) German Holstein cows, but none of the microsatellites revealed any associations. Reddacliff et al. (2005) found an association of one microsatellite allele in SLC11A1 (formerly NRAMP1) with Map resistance in sheep.

Gonda and coworkers (2006) undertook a genome-wide linkage study using ELISA, fecal culture or both to diagnose infected animals. In this study, microsatellites were used to genotype three half-sib families. The number of informative (useful) markers ranged from 151-176 within the three families. Genotypes of “positive” and “negative” animals were pooled and allele frequencies were estimated. Eight chromosomal regions were associated with the pooled samples (bovine chromosomes 7, 10, 12, 14, 15, 18, 20 and 25). The eight chromosomal regions associated with Map infection in pooled genotypes were further tested. Individual genotypes of the daughters were determined for 3-5 microsatellites within 15 cM (an estimated 15 million base pairs) of the markers identified in the pooled samples. Subsequently, only chromosome 20 was found to be linked (P=0.0319) in a chromosome-wide analysis in one of the sire families.

Current management practices are to cull cows after either testing positive for Map or exhibiting clinical signs of the disease. However, clinical signs of Map infection may be delayed as long as four to five years after the initial exposure. Current diagnostic testing has a limited sensitivity for detecting the presence of Map in pre-clinical animals, at which time they are likely to be spreading Map to other animals in the herd through fecal contamination of the environment, food, and water. The low overall sensitivity of ELISA (7-35%) and fecal (38-65%) diagnostic tests and the long incubation period of the disease present major roadblocks to the control of Johne's disease (Whitlock et al. 2000; McKenna et al. 2005; Collins et al. 2006).

Identifying the mutations associated with Map tissue infection will not only provide the opportunity to better understand the genes and gene regulatory elements that are associated with the first event in the pathogenic process that culminates in Johne's disease, but will provide compositions and methods for diagnosis of susceptibility, resistance and/or tolerance to infection by Mycobacterium avium subspecies paratuberculosis (Map).

### SUMMARY OF EXEMPLARY EMBODIMENTS

Applicants herein identify and disclose mutations and genes associated with Map tissue infection, the first event in the pathogenic process that culminates in Johne's disease. Provided are compositions and methods for diagnosis of susceptibility, resistance and/or tolerance to infection by Mycobacterium avium subspecies paratuberculosis (Map).

Loci associated with Mycobacterium avium subspecies paratuberculosis (Map) infection status in US Holsteins were identified using a whole genome SNP (single nucleotide polymorphism) assay (Illumina BovineSNP50 BeadChip™). Two hundred forty-five cows from dairies in New York, Pennsylvania, and Vermont were followed to culling between January 1999 and November 2007 and subsequently were assessed for the presence of Map in both fecal samples and necropsy tissue. An animal was considered tissue-infected if any sample contained at least one colony-forming unit per gram of tissue (cfu/g) and the same definition was employed for fecal samples ach animal was genotyped with the Illumina BovineSNP50 BeadChip. After quality assurance filtering, 218 animals and 45,683 SNPs remained. Genetic loci associated with the following four different case/control classifications were identified: presence of Map in the tissue; presence of Map in feces; presence of Map in both tissue and feces; and presence of Map in tissue but not feces. A case-control genome wide association study (GWA) was conducted to test the four different classifications of Map infection status (Cases) when compared to a Map negative control group (Control). Regions on chromosomes 1, 5, 7, 8, 16, 21, and 23 were identified that showed a moderate significance (P<5×10⁻⁵). Regions on chromosomes 3 and 9 were identified with a high level of association to the presence of Map in tissue and both tissue and feces, respectively (P<5×10⁻⁷, genome-wide Bonferonni P<0.05).

In particular preferred aspects, a 255,586 by region (SEQ ID NO:1) of bovine chromosome 3, referred to herein at the inventive “Map infection linkage disequilibrium region” is disclosed to be associated with Map tissue infection (P=7×10⁻⁸), and is further characterized as including three functionally relevant genes: HIVEP3 (Human immunodeficiency virus type I enhancer-binding protein 3) (SEQ ID NO:2); EDN2 (Endothelin 2) (SEQ ID NO:3); and LOC521287 (Bos taurus similar to forkhead box O6) (SEQ ID NO:4), and the respective coding transcripts (SEQ ID NOS:5, 6 and 7, respectively), and polypeptides (SEQ ID NOS:8, 9 and 10, respectively).

Particular aspects provide genetic markers (single nucleotide polymorphisms or SNPs) identified within a 76 kb region (SEQ ID NO:46) on chromosome 3 that can be used to predict resistance to Map tissue infection in Holstein cows (P=7×10⁻⁸, P=9×10⁻⁷, P=1.2×10⁻⁵, P=0.0006), and thus resistance of animals to Johne's disease.

Certain aspects provide a method for determining at least one of susceptibility, resistance or tolerance to Mycobacterium avium subspecies paratuberculosis (Map) infection of a subject, comprising: obtaining a biological sample from a mammalian subject; determining, using the biological sample, a presence or absence of at least one mutation, or the genotype of at least one single nucleotide polymorphism (SNP) within Map infection linkage disequilibrium region SEQ ID NO:46 that segregates with resistance and/or tolerance to Map tissue infection, or determining a presence or absence of at least one mutation or a genotype of at least one SNP within a Map infection linkage disequilibrium region (SEQ ID NO:1) that is in linkage disequilibrium with the presence or absence of the at least one mutation or with the genotype of the at least one SNP within SEQ ID NO:46; and determining, based thereon, at least one of susceptibility, resistance or tolerance of the mammalian subject to Mycobacterium avium subspecies paratuberculosis (Map) infection.

Additional aspects provide a method for determining at least one of susceptibility, resistance or tolerance to Mycobacterium avium subspecies paratuberculosis (Map) infection of a subject, comprising: obtaining a biological sample from a mammalian subject; analyzing, using the biological sample, a mammalian genotype to determine if the genotype comprises at least one single nucleotide polymorphism (SNP) associated with at least one of susceptibility, resistance or tolerance to Mycobacterium avium subspecies paratuberculosis (Map), wherein the SNP is at least one selected from the SNP group consisting of, relative to the sequence of accession no. NC_{—}007301.3, SNPs at positions 111,606,781, 111,610,137, 111,611,773, 111, 623,092 and 111,682,511 (ARS-BFGL-NGS-113303); and determining, based thereon, at least one of susceptibility, resistance or tolerance of the mammalian subject to Mycobacterium avium subspecies paratuberculosis (Map) infection.

Further aspects provide a method for determining at least one of susceptibility, resistance or tolerance to Mycobacterium avium subspecies paratuberculosis (Map) infection of a subject, comprising: obtaining a biological sample from a mammalian subject; determining, using the biological sample, a presence or absence of at least one mutation, or the genotype of at least one single nucleotide polymorphism (SNP) within at least one gene selected from the group consisting of HIVEP3 (Human immunodeficiency virus type I enhancer-binding protein 3) (SEQ ID NO:2); EDN2 (Endothelin 2) (SEQ ID NO:3); and LOC521287 (Bos taurus similar to forkhead box O6) (SEQ ID NO:4) that segregates with resistance and/or tolerance to Map tissue infection, or determining a presence or absence of at least one mutation or a genotype of at least one SNP within a Map infection linkage disequilibrium region (SEQ ID NO:1) that is in linkage disequilibrium with the presence or absence of the at least one mutation or with the genotype of the at least one SNP within within at least one gene selected from the group consisting of HIVEP3 (Human immunodeficiency virus type I enhancer-binding protein 3) (SEQ ID NO:2); EDN2 (Endothelin 2) (SEQ ID NO:3); and LOC521287 (Bos taurus similar to forkhead box O6) (SEQ ID NO:4); and determining, based thereon, at least one of susceptibility, resistance or tolerance of the mammalian subject to Mycobacterium avium subspecies paratuberculosis (Map) infection.

In particular embodiments of the above methods, the at least one single nucleotide polymorphism (SNP) within Map infection linkage disequilibrium region SEQ ID NO:46, is at least one selected from the SNP group consisting of, relative to the sequence of accession no. NC_{—}007301.3, SNPs at positions 111,606,781, 111,610,137, 111,611,773, 111, 623,092 and 111,682,511 (ARS-BFGL-NGS-113303) as defined herein. In certain aspects, the genotypes of at least two single nucleotide polymorphisms (SNPs) within Map infection linkage disequilibrium region SEQ ID NO:46, are determined. In particular embodiments, at least one of resistance or tolerance of the mammalian subject to Mycobacterium avium subspecies paratuberculosis (Map) infection is determined.

In certain aspects of the above methods, the mammalian subject is bovine.

Particular aspects of the above methods further comprise at least one of culling, selecting, or breeding, based on the determining of at least one of susceptibility, resistance or tolerance of the mammalian subject to Mycobacterium avium subspecies paratuberculosis (Map) infection.

Particular aspects of the above methods further comprise selective breeding to produce offspring having at least one of susceptibility, resistance or tolerance to Mycobacterium avium subspecies paratuberculosis (Map) infection.

### BRIEF SUMMARY OF THE DRAWINGS

FIG. 1 shows a Q-Q plot for p-values from a 1-df test for association (allelic) versus expected for an association of loci with Map infected tissue (tissue positive versus tissue negative). The Q-Q plot shows little to no evidence of a deviation from the expected null distribution of p-values and therefore no evidence of population substructure.

FIG. 2 shows a Q-Q plot for p-values from a 1-df test for association (allelic) versus expected for an association of loci with Map fecal positive (fecal positive versus fecal negative). The Q-Q plot shows evidence of a deviation from the expected null distribution of p-values and therefore evidence of population substructure.

FIG. 3 shows a Q-Q plot for stratified test of association (2×2×4 CMH test) within herd versus expected for an association of loci with Map fecal positive (fecal positive versus fecal negative). The Q-Q plot shows little to no evidence of a deviation from the expected null distribution of p-values and therefore no evidence of population substructure.

FIG. 4 shows a Q-Q plot for 1-df test for association (allelic) versus expected for an association of loci with Map tissue infected, fecal negative (infected) cases versus controls. The Q-Q plot shows little to no evidence of a deviation from the expected null distribution of p-values and therefore no evidence of population substructure.

FIG. 5 shows a Q-Q plot for p-values from a 1-df test for association (allelic) versus expected for an association of loci with the Map tissue infected, fecal positive (clinical) cases versus controls. The Q-Q plot shows evidence of a deviation from the expected null distribution of p-values and therefore evidence of population substructure.

FIG. 6 shows a Q-Q plot for the stratified test of association (2×2×4 CMH test) within herd versus expected for an association of loci with the Map tissue infected, fecal positive (clinical) cases versus controls. The Q-Q plot shows little to no evidence of a deviation from the expected null distribution of p-values and therefore no evidence of population substructure.

FIG. 7 shows a tolerance distribution using peak (left panel) and average (right panel) fecal CFUs and peak (left panel) and average (right panel) tissue CFUs taken at slaughter. The tolerance index was defined as the fecal CFUs (CFU_{f})+100 divided by the tissue (CFUₜ)+100.

FIG. 8 shows a multidimensional scaling (MDS). MDS provides a spatial representation of the relationship between animals. MDS plots also are useful for detecting underlying substructure in the data. In this experiment, the MDS plot did not identify a population substructure.

FIG. 9 shows a genome-wide plot of −log₁₀ (p-values) for an association of loci with tolerance index calculated using the peak fecal divided by peak tissue. Chromosomes 1 through 29 and Chromosome X are shown.

FIG. 10 shows a genome-wide plot of −log₁₀ (p-values) for an association of loci with tolerance index calculated using the average fecal divided by average tissue. Chromosomes 1 through 29 and Chromosome X are shown.

FIG. 11 shows a Q-Q plot of an association analysis with tolerance as a quantitative trait. In panel 5a the peak fecal CFUs is divided by the peak tissue CFUs. The results indicate that there was no evidence of population stratification (λ_{GC}=1.03). Panel 5b shows the results of an average fecal CFUs divided by average tissue CFUs showing no evidence of population stratification (λ_{GC}=1.00).

FIG. 12 shows a genome-wide plot of −Log 10(P-values) for 45,683 SNPs tested for an association with Mycobacterium avium subspecies paratuberculosis (Map) infected tissue (tissue positive compared to tissue negative). Chromosomes 1-29 are shown. The horizontal dark grey line is drawn at −log 10 (5×10⁻⁵) and the horizontal black line is drawn at −log 10 (5×10⁻⁷) to show those significant at the moderate and strong levels of significance, respectively. (Settles et al. 2009)

FIG. 13 shows the locations of single nucleotide polymorphisms (SNPs) (in base pairs) on bovine chromosome 3. The SNP at 111,682,510 (SS86341066) (in bold) was the most strongly associated with tissue infection (P=3×10⁻⁷).

FIG. 14 shows an association analysis of an 82 kb region of chromosome 3. Location of SNPs in basepairs are listed on the x axis and the −log 10 (p value) of the association of each SNP with Map tissue infection is listed on the y axis. A −log 10 value of 1.2 represents P=0.05. SNPs with the greatest evidence for association with Map tissue infection include the SNP at 111,623,092 (P=9.4×10⁻⁷), the adjacent 3′ SNP 111,693,185 (P=1.1×10⁻⁵) and the SNP located between HIVEP3 and EDN2 at 111,682,510 by (P=7.5×10⁻⁸). SNPs that are marked with ** indicate SNPs that were first analyzed with the use of the Illumina bovine SNP50 BeadChip and were also included in the fine mapping of additional SNPs in this region.

FIG. 15 shows a schematic of the location of the genetic markers, and their alleles to form a haplotype over an 86 kb region on bovine chromosome 3. The haplotype shown was only seen in animals with Map tissue infection. Similarly, six haplotypes were only seen in cows without Map tissue infection (23%). Bolded alleles indicate markers found to exclusively segregate with resistance to Map tissue infection.

FIG. 16 shows a multidimensional scaling (MDS) plot. MDS plots provide spatial representations of data that can facilitate interpretation and reveal structural relationships in the data. This MDS plot identified 15 animals (grey) whose multilocus genotypes differed significantly from the other animals allowing these animals to be removed from further analysis.

FIG. 17 shows a genome-wide plot of −log₁₀(p-values) for an association of loci with Map infected tissue (tissue positive versus tissue negative). Chromosomes 1 through 30 and Chromosome X are shown separated by grey scale. The horizontal dark grey line is drawn at −log 10 (5×10⁻⁵) and the horizontal black line is drawn at −log 10 (5×10⁻⁷) to show those significant at the moderate and strong levels of significance, respectively.

FIG. 18 shows genome-wide plot of −log₁₀(p-values) for an association of loci with Map tissue infected and fecal positive phenotype (clinical). Chromosomes 1 through 30 and Chromosome X are shown separated by grey scale. The horizontal dark grey line is drawn at −log 10 (5×10⁻⁵) and the horizontal black line is drawn at −log 10 (5×10⁻⁷) to show those significant at the moderate and strong levels of significance, respectively.

FIG. 19 shows a schematic of the region from chromosome 3 of Bos taurus from position 11432282-111943455 corresponding to NC 007301.3 (based on Btau_{—}4.0).

### DETAILED DESCRIPTION OF EXEMPLARY EMBODIMENTS

A whole genome association analysis identified markers and loci associated with Mycobacterium avium subsp. Paratuberculosis infection status in U.S. Holstein cattle.

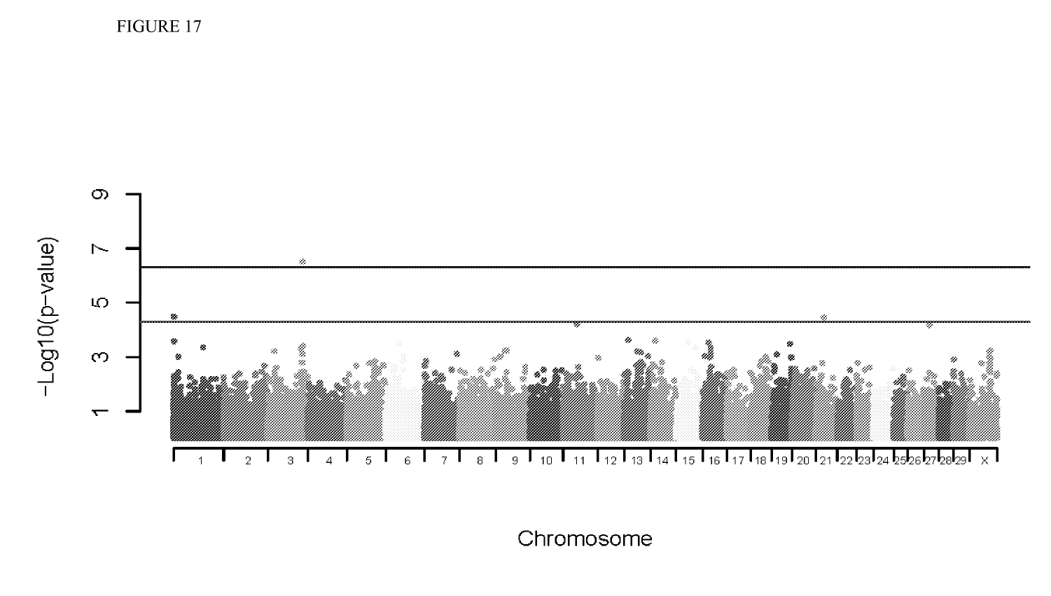

As disclosed under working EXAMPLE 1 herein below, a GWA study was conducted to identify loci associated with Map infection status using four different case/control classifications of the samples: presence of Map in tissue; presence of Map in feces; presence of Map in tissue, but not feces; and the presence of Map in both tissue and feces. Results from the first classification, presence of Map in tissue, produced a single strong association ARS-BFGL-NGS-113303 (MAF=0.36, Bonferroni p=0.014) with resistance to Map penetration of the tissue and 4 moderate associations (FIG. 17). RefSeq genes located within 1 Mb of ARS-BFGL-NGS-113303 identified the gene endothelin 2 (EDN2) located 31 Kb downstream from ARS-BFGL-NGS-113303. EDN2 has been found to be highly expressed in the gastrointestinal tract of the mouse. Takizawa et al. (2005) found that in intestinal epithelial cells EDN2 could be secreted into the lamina propria and the dome region in Peyer's patch, and that it might modulate immune cells for mucosal defense. Further, McCartney et al. (2002) found EDN2 to be highly expressed in mucosal biopsies of humans, including samples taken from patients with inflammatory bowel disease (i.e., Crohn's disease). However, no associations were found between human IBD and mRNA expression.

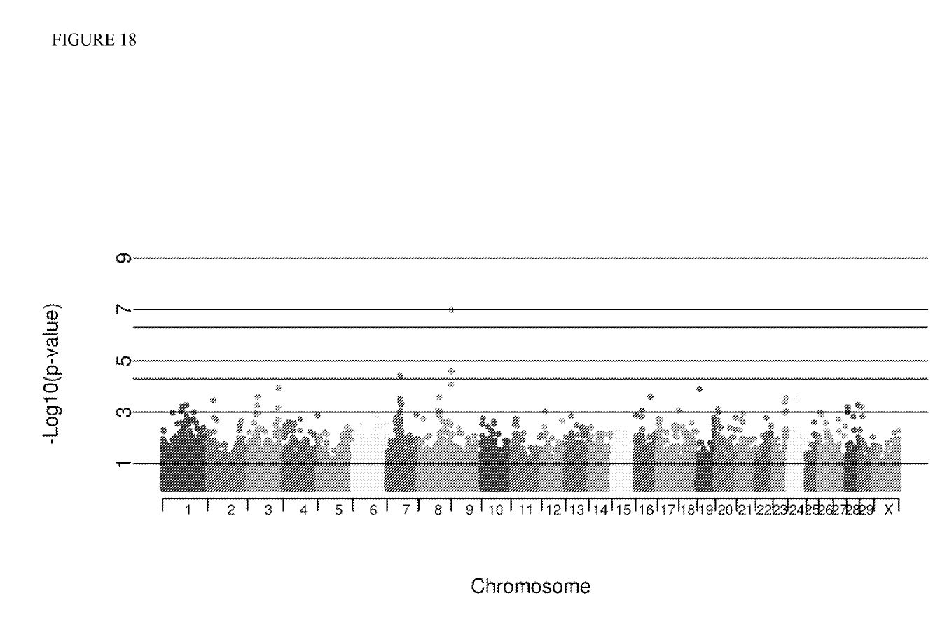

Association with the presence of Map in feces, however, did not yield any strong associations and only 4 moderate associations, 3 of which were on different chromosomes to those identified as associated with Map infection of tissue. The fourth locus, on chromosome 5, was 30 Mb from the SNP found to be moderately associated with Map infection of tissue. Comparing the rank order of SNPs, by significance, for the tissue positive results to the fecal positive results showed the two to have a significantly different significance ordering (data not shown). It is well established that the sensitivity of Map fecal culture in the early stages of infection is low. In this study, fecal culture only detected the presence of Map infection 40% of the time in the presence of tissue infection. This result shows that the differences in sensitivities between the diagnostic tests and the resulting definition of phenotype significantly impacts the loci detected to be associated with Map and their corresponding interpretation. This result led to the question of whether there was a difference in loci associated with a tissue positive, but fecal negative classification and a tissue positive and fecal positive classification. If having fecal culture results provides no additional information, one would expect the rank order of SNPs, by significance, to be similar in these analyses. However, this is not what was found. Results from tissue infection with no fecal shedding did not identify any strongly associated regions and only 3 moderately associated regions, which included the same SNP found to have a strong association (ARS-BFGL-NGS-113303) to tissue positive culture. Results from the analysis of the tissue positive infection in conjunction with fecal shedding phenotype produced one strong association on chromosome 9 (BTB-01957421, MAF=0.01, Bonferonni p=0.005) and 4 moderate associations, 3 of which were neighbors of BTB-01957421 (FIG. 18). There are no Bovine RefSeq genes within 1 Mb of BTB-01957421. The identification of different loci associated with tissue infection, compared to tissue infected and fecal shedding, indicates that there are loci important to different stages of the disease.

The infection of cells by Map is the first step toward Johne's disease, but it may not be a guarantee of disease. Similarly, fecal shedding of Map may occur because of environmental exposure, but without tissue infection the animal may not become diseased. Within hours of Map ingestion, the bacterium attaches to the host's intestinal mucosa and penetrates the mucosa/M cells overlying Peyer's patches (Wu et al. 2007; Momotani et al. 1988). Map that survive phagocytosis by macrophages acquire nutrients for growth and replicate. Map is later disseminated in macrophages and is associated with early paucibacillary lesions. This cell-mediated immunological response restricts the expansion of Map (Clarke 1997; Waters et al. 1999). At this stage, and depending on other factors (such as the dose of Map, the genetics of the host, local tissue cytokine concentrations, stress, and hormone levels of the cow) the animal may either clear Map or may develop a persistent Map infection that culminates in Johne's disease (Harris and Barletta 2001). For those animals that progress to Johne's disease, humoral-mediated responses replace cell-mediated responses. These animals exhibit fecal shedding, positive serological tests, greater Map replication rates, multibacillary lesions, and clinical disease (Whittington and Sergeant, 2001). The very different processes involved in early disease and late disease may also be reflected in the different loci found to be associated with the classification of tissue infection as compared to clinical infection. Because the etiology of the disease is not fully understood, loci associated with tissue infection or the “clinical” diagnosis are not referred to as Johne's susceptibility or resistance loci, as these loci may have different roles in the pathogenesis of Johne's disease at different stages. Therefore, the use of both tissue and fecal diagnostic testing provides a more complete profile of the health status of the cow.

While others have found evidence of association with Johne's disease on bovine chromosome 20 (BTA20), the present study shows no evidence for a locus associated with Johne's disease on BTA 20 in any of the four phenotype classifications analyzed. Additionally, in the Crohn's disease literature two genes consistently have the most significant association with Crohn's: IL23R and NOD2 (also known as CARD15). While others have shown NOD2 to have “no association” with Johne's disease (Taylor et al. 2006), no one has yet looked at the IL23R ortholog on bovine chromosome 3 (located at 83 Mb). Focusing on all SNPs within 1 Mb of both NOD2 and IL23R, no loci in the vicinity of these genes were found to be associated with any Map infection phenotype (data not shown).

As disclosed under working EXAMPLE 1 herein below, the loci associated with tolerance to Johne's disease in cattle were examined. One critical reason for doing genome-wide studies is in the characterization of the phenotype. In working EXAMPLE 2 the phenotype was defined as tolerance to bovine paratuberculosis. The definition of tolerance was that animals that were infected had high doses of Map in the tissues but lower levels of Map in the feces. Once the animal becomes infected it can spread the Map to other animals through the fecal material (Harris and Barletta 2001). The ability to reduce the spreading of the disease by having low doses of Map in the feces, in comparison with the levels of Map in the tissue, was characterized as tolerance. Because tolerance can only be measured in infected animals (tissue positive), and fecal shedding occurs after a long incubation period, the age of infection and infection pressure could play an important role in the definition of tolerance. From the data a small negative correlation was observed between infection and age (r=−0.09) or fecal shedding and age (r=−0.18), indicating no effect of age with disease status. Using the culture of tissue as a method to identify the true disease status of the animals reduced the misclassification of the phenotype in animals with early disease frequently found when animals were diagnosed using fecal culture or ELISA (Sockett et al. 1992, Whitlock et al. 1996, 2000).

Traits, including most diseases, are distributed as quantitative traits and usually determined by complex genetic and environmental factors and potentially gene-gene interactions and gene-environment interactions (Li et al. 2006). In many cases of complex diseases, the infectious status of the animal is characterized as some threshold value (Churchill and Doerge, 1994). The use of genome-wide association studies with a quantitative trait provides relevant information of genetic variation associated with disease. A limiting factor often faced in the case-control association studies is the limited sample size. One alternative to reduce the effects of the small number of cases and controls is to analyze tolerance as a quantitative trait, in this case including all the positive animals with a tolerance index. The peak CFUₜ and average CFUₜ were highly correlated with peak CFU_{f} and average fecal CFU_{f} (r=0.73 and r=0.79, respectively).

In a review article, Abubakar and colleagues (2008) suggested that there is sufficient evidence for the presence of Mycobacterium avium subspecies paratuberculosis in the gut of patients with Crohn's disease. A summary risk difference of 0.23 (95% CI, 0.14-0.32) was calculated, which remained consistent after sensitivity analysis excluding various sets of studies, and suggested an association with Johne's disease. Consequently, the persistence of intolerant animals on the farm will help to spread the disease and consequently may increase the incidence of Crohn's disease in humans. None of our SNPs were located close to genomic regions known to be associated with Crohn's disease in humans. However, one SNP located on BTA 15 was localized 522 kb from the gene Interleukin-18 (IL18), which is a proinflammatory cytokine and considered to be a key factor in inflammatory bowel diseases (IBD) (Banerjee and Bond, 2008). This SNP is also located 428 kb from the PTS gene. Mutations in PTS result in serotonin and catecholamine deficiency that affect a range of immune responses involving cytokines, lytic activity, and antibody production (Thony and Blau 1997, Alaniz et al. 1999).

The SNP associated with T_{Average} residing on BTA 2 is located 45 kb from CXCR4, and 188 Kb from MCM6. The inhibition of the activity of CXCR4 with AMD3100 decreases invasion of human colorectal cancer cells in vitro (Li et al. 2008). MCM6 has been associated with lactose intolerance and also with the recurrence of squamous papillary tumor (SP) (Xu et al. 2007; Imtiaz et al. 2007).

According to particular aspects, the identification of loci associated with tolerance to Johne's disease can be used as a new prediction method to select animals to reduce spreading of Mycobacterium avium subspecies paratuberculosis in the environment and reducing the incidence and losses caused by Johne's disease.

### Example 1

### Loci Associated with Mycobacterium Avium Subsp. Paratuberculosis (Map) Infection Status in U.S. Holstein Cattle were Identified by Whole Genome Association Analysis

Overview. The experiments and data disclosed in this working EXAMPLE 1 identify loci associated with Mycobacterium avium subspecies paratuberculosis (Map) infection status in US Holsteins. The data were obtained using the Illumina BovineSNP50 BeadChip™ whole genome SNP (single nucleotide polymorphism) assay.

The population of animals used in this study was Holstein cows from four geographically distinct herds culled for any reason, including having a Map positive diagnostic test. Specifically, two hundred forty-five cows from dairies in New York, Pennsylvania, and Vermont were followed to culling between January 1999 and November 2007 and subsequently were assessed for the presence of Map in both fecal samples and necropsy tissue. An animal was considered tissue-infected if any sample contained at least one colony-forming unit per gram of tissue (cfu/g) and the same definition was employed for fecal samples. Each animal was genotyped with the Illumina BovineSNP50 BeadChip. After quality assurance filtering, 218 animals and 45,683 SNPs remained. Genetic loci associated with the following four different case/control classifications were identified:

presence of Map in the tissue;

presence of Map in feces;

presence of Map in both tissue and feces; and

presence of Map in tissue but not feces.

A case-control genome wide association study (GWA) was conducted to test the four different classifications of Map infection status (Cases) when compared to a Map negative control group (Control). Regions on chromosomes 1, 5, 7, 8, 16, 21, and 23 were identified that showed a moderate significance (P<5×10⁻⁵). Regions on chromosomes 3 and 9 were identified with a high level of association to the presence of Map in tissue and both tissue and feces, respectively (P<5×10⁻⁷, genome-wide Bonferonni P<0.05).

Determination of the presence of Map in tissue and/or feces. Map infection status was evaluated from culture diagnostic testing of fecal samples and necropsy tissues. Genetic loci associated with the above four different case/control classifications were searched. The first classification tested if there were loci associated with a tissue positive result for Map, regardless of fecal status. These loci are associated with the bacterium's ability to infect the host's cells.

The second classification tested for loci associated with fecal shedding, which may be independent of those associated with Map tissue infection and may represent loci responsible for a cow's ability to transmit Map through its feces. Further, this analysis is comparable to studies that inferred disease status by the presence of fecal Map, in the absence of tissue data.

When considering both fecal and tissue results to classify animals, four possible subgroups arise:

Map negative controls (fecal−, tissue−);

fecal shedding but tissue negative (fecal+, tissue−);

tissue infected but not fecal shedding (fecal−, tissue+); and

“clinical” (fecal+, tissue+).

Genome wide association case-control association tests were performed for loci associated with the following subgroups: tissue infected but not fecal shedding animals (cases) relative to negative animals (controls), and “clinical” animals (cases) relative to negative animals (controls). These two classifications were expected to identify loci associated with tissue-infected animals that were not fecal shedding as compared to “clinical” animals, and thus to allow determination of whether any loci are shared. According to particular aspects, the identification of loci associated with each of these phenotypes can be used to develop a marker-assisted selection program to aid in the control of Johne's disease. Moreover, identifying Map-tolerant or resistant genotypes represents a new way of evaluating the impact of disease on animals. Selection of animals for tolerance would put different pressures on the host (cattle) and the pathogen (Map) and may be more or less effective as a means of controlling Johne's disease than selection for disease resistance.

### Materials and Methods:

Study Population and Phenotypes. Two hundred and forty-five Holstein cows from dairies in New York (Herd A), Pennsylvania (Herds B and D), and Vermont (Herd C) were followed to culling between January 1999 and November 2007 and subsequently were assessed for the presence of Map in both fecal and necropsy tissue. Tissue samples from the ileum, ileo-cecal valve, and two adjacent ileo-cecal lymph nodes were cultured for the presence of Map following the protocols previously described in Whitlock et al. (1996, 2000). In addition, fecal samples were taken at necropsy and were cultured for the presence of Map using the same procedure. Each animal was considered tissue-infected if any cultured sample from any of the four tissues had greater than zero colony-forming units per gram of tissue (cfu/g). Fecal culture status was similarly classified. Ninety-five animals were classified as tissue positive, and 45 animals were classified as fecal positive. As expected, the sensitivity of the culture of tissue was significantly greater than the fecal culture in detecting the presence of Map. Within the tissue positive samples, fecal culture detected the presence of Map in only 40% of the samples (38 out of 94, when both tissue and fecal results were present, one tissue positive sample had a missing fecal sample). Six animals tested positive in fecal culture but not in tissue culture. The fecal cfu/g values in these animals were less than five and may represent what many consider to be fecal “pass-through”, which is when an animal has a fecal positive test due to environmental conditions but is not Map infected. The distributions of the fecal and tissue results by herd are shown below in Tables 1a and 1b. Each sample was further characterized for potential confounding variables such as age at culling (median 56.5 months of age, quantiles 1 and 3 equal to 44.3 and 72.25 mo., respectively) and herd of origin.

TABLE 1a
Map tissue culture status by herd before
quality control filtering of the data.

| Data Set | Negative | Positive | Unknown | Total |
|---|---|---|---|---|
| Herd A | 45 | 30 | 1 | 76 |
| Herd B | 40 | 9 | 0 | 49 |
| Herd C | 14 | 17 | 0 | 31 |
| Herd D | 38 | 39 | 12 | 89 |
| Totals | 137 | 95 | 13 | 245 |

Tissue positive status was determined as at least one sample from any of 4 tissues yielding at least one colony forming unit of Map (peak cfu/g > 0).

TABLE 1b
Map fecal culture status by herd before
quality control filtering of the data.

| Data Set | Negative | Positive | Unknown | Total |
|---|---|---|---|---|
| Herd A | 71 | 4 | 1 | 76 |
| Herd B | 47 | 2 | 0 | 49 |
| Herd C | 19 | 11 | 1 | 31 |
| Herd D | 51 | 28 | 10 | 89 |
| Totals | 188 | 45 | 12 | 245 |

Fecal positive status was determined as at least one fecal sample containing at least one colony forming unit of Map (peak cfu/g > 0).

Genotyping. DNA was extracted from the tissue of each animal using the Puregene DNA extraction kit per manufacturer's instructions (Gentra, Minneapolis, Minn.). Sample DNA was quantified and genotyped using the Illumina BovineSNP50 BeadChip™ (Matukumalli et al. 2008). The Illumina BovineSNP50 BeadChip™ assay contains 53,243 SNPs with a mean spacing of one SNP every 49.4 kb (median spacing of 37 kb; quartiles 1 and 3 equal to 27.6 kb and 54 kb, respectively; and a maximum distance of 1.45 Mb). The BovineSNP50 BeadChip™ also contains an additional 1,828 SNPs, which are located on contigs that are not mapped to a chromosome (Chr. Un). All samples were brought into a single BeadStudio™ file, and genotypes were identified using a custom genotype clustering file developed at the University of Missouri using more than 7,000 samples from multiple Bos taurus cattle breeds.

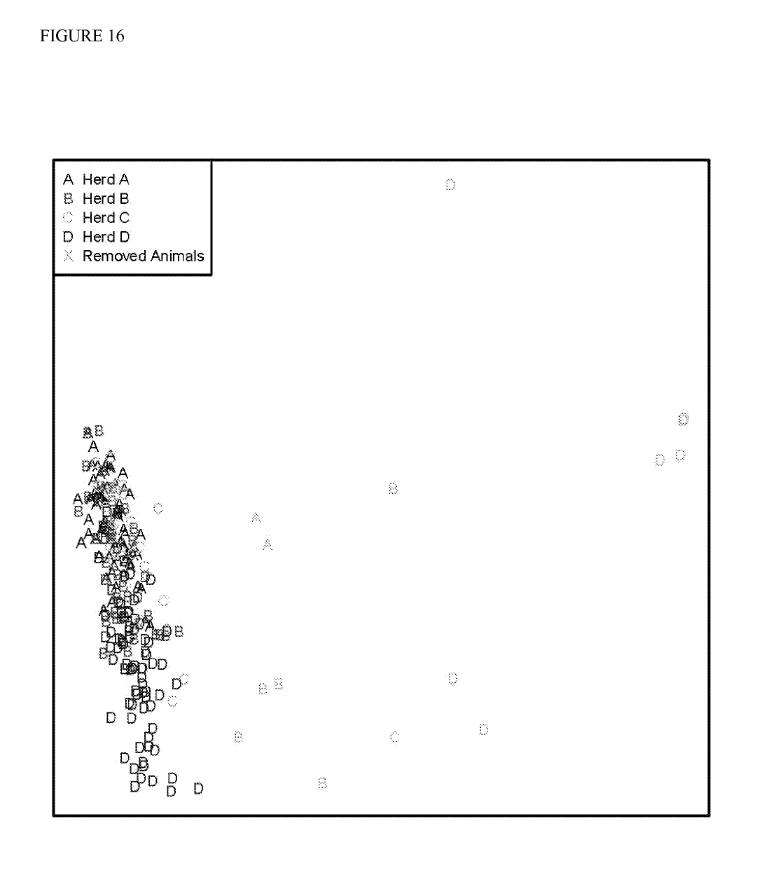

Quality Assurance. Seven animals (one from Herd A, five from Herd B and one from Herd D) were excluded from the analysis for quality with a genotype no-call rate greater than 10%. An excess no-call rate is an indicator of low quality DNA. To assess technical variation, one animal was hybridized to two arrays, resulting in a greater than 99% identity of called genotypes (2 mismatches). Multi-dimensional scaling (MDS) of the matrix of genome-wide identity-by-state (IBS) distances was used to provide a two-dimensional projection of the data onto axes representing components of genetic variation. Animals whose genetic ancestry differs significantly appear as outliers on the MDS plot. To avoid confounding the multi-dimensional scaling by extended linkage disequilibrium, we thinned the genotype data to a set of 10,098 SNPs, in which no pair of SNPs was correlated with r²>0.2. For this set of SNPs, the genome-wide IBS pair-wise identities between each pair of animals was computed using PLINK (Purcell et al. 2007; Version 1.04). These IBS-relationships were converted to genetic distances by subtracting them from one, and the matrix of pair-wise IBS distances was used as input for multi-dimensional scaling. The projection of the data onto the first two multi-dimensional scaling axes is shown in (FIG. 16). The multidimensional scaling (MDS) plot provides a spatial representation of data to facilitate interpretation and reveal structural relationships in the data. The MDS analysis identified fifteen animals that were clearly distinct from the majority of animals, two animals from Herd A, five animals from Herd B, one from Herd C and seven animals from Herd D. These animals were removed from further analysis, resulting in a substantial reduction of the genomic inflation factor (based on median chi-squared, data not shown).

After removing animals for genotype quality and excessive genomic variance, the age ranges of the remaining control animals (tissue negative) were compared to those for tissue Map positive animals. The mean age of control animals was 58.1 months, while the mean age of the tissue Map positive animals was 60.5 months of age. Five control animals were significantly younger (approximately 12 months of age when culled) than the youngest of the tissue Map positive animals (approximately 23 months of age when culled). These animals were excluded from the study as their youth may have affected the ability to detect Map in their tissues and/or feces. The resulting mean age at culling for control animals (tissue negative) was 60 months, with a range of 21.8 to 144 months and the mean age at culling was 60.5 months for Map tissue positive animals with a range of 22.9 to 135 months. After removing 27 samples for quality, genetic variability and age, 218 animals remained in the study with an average genotype call rate of 98.9%, of which 90 animals tested positive for Map in at least one tissue sample (tissue positive) and 41 animals tested positive for Map in fecal samples (fecal positive). Thirty-five animals tested positive in both fecal and tissue samples (“clinical”), 6 animals tested positive in fecal culture but not tissue (“pass-through”), 54 animals tested positive in at least one tissue sample but not fecal sample (“infected”), 112 samples tested negative for Map in both fecal and tissue samples (“negative”), 11 samples were untested for either fecal or tissue, and seven of these animals were untested for both fecal and tissue samples (FIG. 20). These animals were left in the study to contribute to SNP level quality assurance but did not contribute to any association test statistic. The distribution of animal numbers by herd is presented in Tables 2a and 2b. 1,276 SNPs with >10% genotype no-call rate and 8,317 with a minor allele frequency (MAF)<0.01, of which 6,356 were monomorphic, were excluded. Genome-wide, 45,683 SNPs (82.9%) passed these quality control filters.

TABLE 2a
Map culture of tissue status by herd after
quality control filtering of the data.

| Data Set | Negative | Positive | Unknown | Total |
|---|---|---|---|---|
| Herd A | 43 | 29 | 1 | 73 |
| Herd B | 31 | 8 | 0 | 39 |
| Herd C | 13 | 17 | 0 | 30 |
| Herd D | 32 | 36 | 8 | 76 |
| Totals | 119 | 90 | 9 | 218 |

Tissue positive status was determined as at least one sample in any of 4 tissues containing at least one colony forming unit of Map (peak cfu/g > 0).

TABLE 2b
Map fecal culture status by herd after
quality control filtering of the data.

| Data Set | Negative | Positive | Unknown | Total |
|---|---|---|---|---|
| Herd A | 68 | 4 | 1 | 73 |
| Herd B | 37 | 2 | 0 | 39 |
| Herd C | 18 | 11 | 1 | 30 |
| Herd D | 45 | 24 | 7 | 76 |
| Totals | 168 | 41 | 9 | 218 |

Fecal positive status was determined as at least one fecal sample containing at least one colony forming unit of Map (peak cfu/g > 0).

Statistical Analysis. Standard one-degree-of-freedom (df) allelic, 1-df dominance, 1-df recessive and 2-df (genotypic) tests of association with genotype between cases and controls were calculated. When evidence for stratification occurred, a within-herd Cochran-Mantel-Haenszel (CMH) test 2×2×K (K=4 herds) of association with genotype was performed. All calculations and plots were performed using the R statistical environment and PLINK (Purcell et al. 2007; Version 1.04). For genome-wide association, uncorrected P values less than 5×10⁻⁷ provided strong evidence of association and uncorrected P values between 5×10⁻⁵ and 5×10⁻⁷ were considered to provide moderate evidence (Wellcome Trust Case Control Consortium 2007). Physical positions and alleles were expressed in terms of the forward strand of the reference genome (Baylor College of Medicine Human Genome Sequencing Center Btau 4.0).

Quantile-quantile plots. A quantile-quantile plot (Q-Q plot) is a useful graphical method for visualizing the differences between a random sample from a population and the expected probability distribution under the null hypothesis. In a genome-wide association study, deviation of the sample distribution p-values from the expected null hypothesis distribution p-values can be attributed to extra variance in the test statistics due to population substructure (Devlin & Roeder 1999). In this study, each Q-Q plot compares the chi-squared (or Cochran-Mantel-Haenszel; CMH) p-values for each SNP versus the expected p-value distribution under the null hypothesis of no association at any locus. Each Q-Q plot was then inspected for a significant deviation from the null distribution. In addition, the median of the chi-squared values was calculated and tested for a statistical difference from the expected value under Hₒ. When the Q-Q plot and median chi-square showed evidence of population stratification, stratified analysis was performed, clustering the data within herds and performing a 2×2×4 CMH test. The resulting Q-Q plot and median chi-square was investigated after the stratified analysis to determine if clustering by herd was successful in adjusting for population stratification.

### Results:

A case-control genome wide association study (GWA) was conducted to test four different classifications of Map infection status defined by results from Map culture from both tissue and fecal samples.

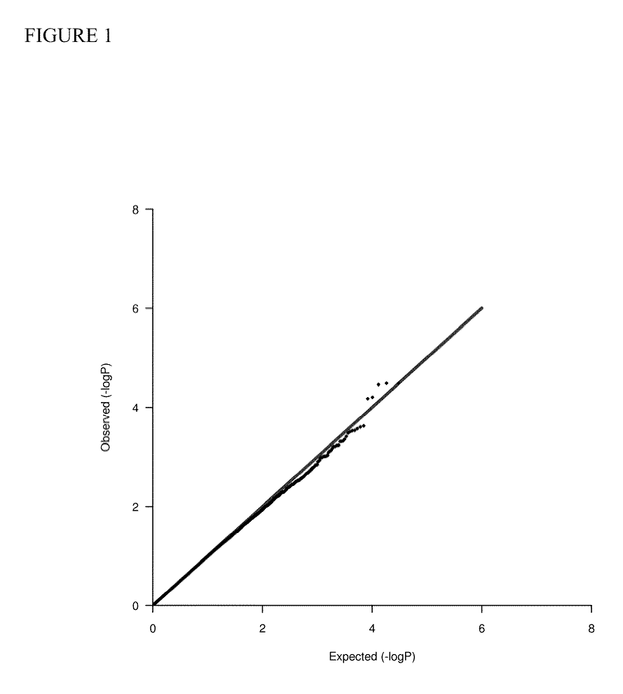

Association of loci with Map infected tissue (tissue positive vs. tissue negative). Single SNP analysis was conducted to test the association of loci with a culture of tissue Map positive result. Cases in this analysis were defined as animals with a Map positive tissue result (n=90) and controls were animals with a Map negative tissue result (n=119) regardless of fecal shedding status. A strong association was found with the ARS-BFGL-NGS-113303 SNP located on BTA 3 using the basic allelic model at position 111,682,510 by (p=3×10⁻⁷; P=0.014 after Bonferonni correction). Seven SNPs were found to be moderately significant (p<5×10⁻⁵) with Map infected tissue, located on chromosomes 1 and 21 for the basic allele frequency difference model and on chromosomes 5 and 16 using the dominance model (Dom; Table 3A). There was no evidence for population substructure on the Q-Q-plot (FIG. 1), or based on the genomic inflation factor (based on median chi-square value, λ_{gc}=1).

Table 3B shows SNPs associated with Map infection status.

TABLE 3A
Genomic regions associated with Map (* Bonferonni significance, when applicable, in
brackets).

| | | | | | Odds | | RefSeq Genes |
|---|---|---|---|---|---|---|---|
| SNP | Chr | Pos (bp) | Phenotype | Test | Ratio | p-value | (1 Mb) |
| Hapmap57114- | 1 | 3,083,368 | Tissue | Allelic | 2.31 | 3.264e−05 | SOD1 |
| rs29012843 | | | | | | | |
| ARS- | 1 | 3,083,498 | Tissue | Allelic | 2.31 | 3.264e−05 | SOD1 |
| USMARC- | | | | | | | |
| Parent- | | | | | | | |
| DQ381153- | | | | | | | |
| rs29012842 | | | | | | | |
| BTB- | 1 | 14,995,903 | Infected | Allelic | 0.31 | 4.269e−05 | NONE |
| 00757888 | | | | | | | |
| ARS-BFGL- | 3 | 111,682,510 | Tissue | Allelic | 0.33 | 3.062e−07 | FOXJ3, EDN2, |
| NGS-113303 | | | | | | *(0.014) | CTPS, CITED4, |
| | | | | | | | NFYC |
| | | | Infected | Allelic | 0.33 | 3.290e−05 | |
| Hapmap27802- | 5 | 73,634,207 | Tissue | Dom | 0.30 | 3.708e−05 | TRA1, ALDH1L2 |
| BTA-49707 | | | | | | | |
| BTA-95292- | 5 | 106,209,963 | Fecal | CMH | 91.2 | 4.163e−05 | T2R67, MAGOHB, |
| no-rs | | | | | | | KLRA1, KLRJ1 |
| ARS-BFGL- | 7 | 47,688,319 | Clinical | CMH | 3.689 | 3.723e−05 | IGH3 |
| NGS-37513 | | | | | | | |
| ARS-BFGL- | 8 | 74,335,842 | Fecal | CMH | 0.30 | 3.389e−05 | STC1 |
| NGS-3865 | | | | | | | |
| BTB- | 9 | 647,609 | Clinical | CMH | 41.50 | 2.511e−05 | None |
| 00478134 | | | | | | | |
| BTB- | 9 | 698,262 | Clinical | CMH | 41.50 | 2.511e−05 | None |
| 00478151 | | | | | | | |
| Hapmap28625- | 9 | 786,610 | Clinical | CMH | 41.50 | 2.511e−05 | None |
| BTA-149369 | | | | | | | |
| BTB- | 9 | 813,310 | Clinical | CMH | NA | 1.029e−07 | None |
| 01957421 | | | | | | *(0.005) | |
| | | | Fecal | CMH | NA | 1.661e−05 | |
| BTB- | 16 | 27,186,964 | Tissue | Dom | 3.71 | 4.632e−05 | PARP-1, RPAC2, |
| 01274618 | | | | | | | PS-2, CABC1, |
| | | | | | | | SCCPDH, DMNT2 |
| BTB- | 16 | 27,237,455 | Tissue | Dom | 3.97 | 2.579e−05 | PARP-1, RPAC2, |
| 01274755 | | | | | | | PS-2, CABC1, |
| | | | | | | | SCCPDH, DMNT2 |
| Hapmap60593- | 21 | 26,660,590 | Infected | Allelic | 5.29 | 8.115e−06 | BCL2A1, |
| rs29025761 | | | | | | | ZFAND6, |
| | | | | | | | MESDC-2, IL16, |
| | | | | | | | MCEE |
| | | | Tissue | Allelic | 4.18 | 3.453e−05 | |
| Hapmap53770- | 23 | 48,404,721 | Fecal | CMH | 3.0730 | 4.392e−05 | EEF1E1 |
| ss46526325 | | | | | | | |

TABLE 3B

SNPs associated with Map infection status

| | | RefSeq Genes | btau40 | | |
|---|---|---|---|---|---|
| Phenotype | p-value | Within 1 Mb | _contig | btau40_pos | Nucleotide Sequence |
| Infection | 3.06E−007 | F0XJ3, EDN2, | 3 | 111682510 | TAAAAAGACAAGAGAC |
| | | CTPS, CITED4, | | | ATCCAGTAGAGGCAACA |
| | | NFYC | | | GGGTGCCCAGGCCTGGG |
| | | | | | CCTCAGGAGA[A/C]AAG |
| | | | | | TTTGGGCTGGAGGTGGA |
| | | | | | CTCTAGCAATCATTGGC |
| | | | | | TAGAGATGGAAATTCAG |
| | | | | | CCTATA |
| | | | | | (SEQ ID NO: 11) |
| Infection | 8.12E−006 | BCL2A1, | 21 | 26660590 | CCTGTAGGATGCACACT |
| | | ZFAND6, | | | GAACTGAGCTGTGGATG |
| | | MESDC-2, IL16, | | | AAGCCCAGAGAAGGTG |
| | | MCEE | | | AATAATTGAC[T/G]CAA |
| | | | | | GGTCACAGGCTTGACAG |
| | | | | | GGTGACACTGAAGTACA |
| | | | | | AACTTTCTTGTCCGGCTC |
| | | | | | TGCTC |
| | | | | | (SEQ ID NO: 12) |
| Disease | 1.03E−007 | None | 9 | 813310 | AGGGGAGAAGACAGAC |
| | | | | | AGAGGATACCCAGCAAT |
| | | | | | GCCAGCTGGGAGGCTGG |
| | | | | | CTCCACCACC[C/G]GGG |
| | | | | | GCTGCTGTTGTCTCTGAT |
| | | | | | CCAGCTTTTCTAGTCTG |
| | | | | | GTGGGATGGGGTAACAT |
| | | | | | CTGTG |
| | | | | | (SEQ ID NO: 13) |
| Disease | 2.51E−005 | None | 9 | 647609 | CTAATCTCTTATTAATG |
| | | | | | GTAGCTATAATACTTTT |
| | | | | | ATATTTAGGACTTTCAG |
| | | | | | AAAAACCTG[A/G]TCAA |
| | | | | | AGTCAACAATTTCAGCA |
| | | | | | TTATCTTGGGGAAACTG |
| | | | | | TGTGTGAATGAGCTCCA |
| | | | | | CTGCC |
| | | | | | (SEQ ID NO: 14) |
| Disease | 2.51E−005 | None | 9 | 698262 | GGGGATTTTTCAGTAAG |
| | | | | | AGGTTAATAACCAGTGA |
| | | | | | GGTAAACATGTATAACA |
| | | | | | ACCTGCTTA[T/C]ATGCA |
| | | | | | CACTGGGAAGTGTCATG |
| | | | | | AACCTTTCTCACCTCCAT |
| | | | | | GCTGAGTTTCCAGAGAA |
| | | | | | CCA |
| | | | | | (SEQ ID NO: 15) |
| Disease | 2.51E−005 | None | 9 | 786610 | ATACTGTCAGAGCAGGC |
| | | | | | ATCTCAGAATCAGACAC |
| | | | | | TTTACCTGATCCCTCTCT |
| | | | | | TACAGAGT[C/G]TGAAA |
| | | | | | CAGCTTTCAAGTATTGG |
| | | | | | GAAGAGGTAGATGCTCC |
| | | | | | CCATACCAAAAGCATCC |
| | | | | | CTGC |
| | | | | | (SEQ ID NO: 16) |
| Infection | 2.58E−005 | PARP-1, RPAC2, | 16 | 27237455 | AAAAAGTAGAGTGATG |
| | | PS-2, CABC1, | | | ACAGACCCATTATATAA |
| | | SCCPDH, | | | TTGAAACATGTTATAAA |
| | | DMNT2 | | | GCCTCAACAA[A/G]TAA |
| | | | | | CACATGAATAAGAAGTC |
| | | | | | CAGAAATGGGGTCAGTT |
| | | | | | ACATAAAGGAATTTAAA |
| | | | | | ATTTAC |
| | | | | | (SEQ ID NO: 17) |
| Infectoin | 4.63E−005 | PARP-1, RPAC2, | 16 | 27186964 | GATGAGATGCATCGAAA |
| | | PS-2, CABC1, | | | ACTGCAATGCCGGCAGC |
| | | SCCPDH, | | | TAGCACTGGTCAACAGA |
| | | DMNT2 | | | AAGGGCCCA[A/G]TTCTC |
| | | | | | CATGCTAATGCCCGATT |
| | | | | | GCACAATCAATGCTTCC |
| | | | | | CAAGTTGAAGGAATTAG |
| | | | | | GCTA |
| | | | | | (SEQ ID NO: 18) |
| Infection | 3.26E−005 | SOD1 | 1 | 3083368 | ATCTGCTTGGATCTTCTC |
| | | | | | ATTAGCGAATTCCAAGA |
| | | | | | AGATGAGCCAGATCAGC |
| | | | | | CCAGGGAC[T/C]GTGAA |
| | | | | | GATCCCAGAGGAGGAC |
| | | | | | ACACACTCCCATCCTGT |
| | | | | | GAGCATTTTTCTTATGTG |
| | | | | | TCAA |
| | | | | | (SEQ ID NO: 19) |
| Infection | 3.26E−005 | SOD1 | 1 | 3083498 | tctgagtgttcattgctgcaccgcctctc |
| | | | | | caagctcccaagggcatctctctacgta |
| | | | | | ata[T/G]taatgcctggccaggtccc |
| | | | | | atccattctgacagacaaaggacaRR |
| | | | | | cgctactttacattt |
| | | | | | (SEQ ID NO: 20) |
| Disease | 3.39E−005 | STC1 | 8 | 74335842 | ACCTGATGACTATTAAG |
| | | | | | AAATTAACACGTAATCT |
| | | | | | ACATGACTGGCCAGCAG |
| | | | | | CCCCACTCA[A/G]GGAG |
| | | | | | TTCCTTACTCCTAAGCA |
| | | | | | GTTCTTAGGAGTTAGAA |
| | | | | | CTCTTCAGTTCAGTTCA |
| | | | | | GTTGC |
| | | | | | (SEQ ID NO: 21) |
| Infection | 3.71E−005 | TRA1, ALDH1L2 | 5 | 73634207 | AATAACTTAAAAATTAT |
| | | | | | TGAAGTGTAGTTTATGT |
| | | | | | ACAATGTAATGTTAATT |
| | | | | | TCATATGTA[T/C]GGCAA |
| | | | | | AACCACTCAGTTATACA |
| | | | | | GATATGTATACATGTAT |
| | | | | | ACTAGAAAAGAACCATT |
| | | | | | CTTA |
| | | | | | (SEQ ID NO: 22) |
| Disease | 3.72E−005 | IGH3 | 7 | 47688319 | ATTCTCTCTGGTCATGG |
| | | | | | CTATTCATTCGTCCCTTC |
| | | | | | CTACCTACACTTGCCCT |
| | | | | | GTTCCAGG[T/C]TGGCCA |
| | | | | | TCCTTCTCTCCATCTGCC |
| | | | | | CATTTCCTCTGGAAAAG |
| | | | | | CAACCACCTTGCAGAGA |
| | | | | | AG |
| | | | | | (SEQ ID NO: 23) |
| Disease | 4.16E−005 | T2R67, | 5 | 106209963 | ACACTTTCCAGTTTTAG |
| | | MAGOHB, | | | ATATGCTCATATAGCAA |
| | | KLRA1, KLRJ1 | | | TGCTTCCATATCTAAGC |
| | | | | | TTTAGTCTC[T/C]TGAAG |
| | | | | | GGTTTGTTGTTGTTGTTC |
| | | | | | AATCACCTAGTCATGTC |
| | | | | | CAACTCTGCAATCCCAT |
| | | | | | GGA |
| | | | | | (SEQ ID NO: 24) |
| Infection | 4.27E−005 | NONE | 1 | 17106221 | ATGTCTTGAGTAATACT |
| | | | | | TTTACTTGTTCAGAAAG |
| | | | | | TTTATTTGTTTATTTTCC |
| | | | | | ATAATGTA[T/C]GTACTG |
| | | | | | CCAGAATGTTACTCAGT |
| | | | | | ATACTCTTGTATCTATCA |
| | | | | | AACTTTCAGCAGTTAGG |
| | | | | | TC |
| | | | | | (SEQ ID NO: 25) |
| Disease | 4.39E−005 | EEF1E1 | 23 | 48404721 | GGCCCACGAGTCCTAGC |
| | | | | | CAGGTGGCCTTGCCTTG |
| | | | | | TGGCCCTGTTGAGTTGC |
| | | | | | TTTGATCAC[A/G]TGACT |
| | | | | | TGTTCATACGTGGCTGG |
| | | | | | TCCATAGGTTGGAGCAG |
| | | | | | ATTTCACACACAAACCC |
| | | | | | CTGA |
| | | | | | (SEQ ID NO: 26) |
| Qualitative | 1.83E−007 | PTS, IL18, | 15 | 21254062 | AGGGAGGGTTTGGCACT |
| (Average)/ | | TEX 12, | | | GGCAAGGGGTCCATGGA |
| Qualitative | | BCO2, PIH1D2,T1 | | | ACATGGACAGAATGAA |
| (Peak) | | MM8B, CRYAB, | | | GCTCTGGGAG[T/G]GAG |
| | | HSPB2, NCAM1, | | | GGGCAGAAGCACATGA |
| | | DR2 | | | GGAGGTCAAGCTTCAGG |
| | | | | | CCCTTCACCTTGACACA |
| | | | | | ATGGGAA |
| | | | | | (SEQ ID NO: 27) |
| Quantitative | 4.23E−007 | SLC35F5, | 2 | 68641435 | CAAAGATTTTTGGTCTG |
| | | ACTR3, LYPD1 | | | ATATAAATGTGTGTCTT |
| | | | | | CTTTTTAGGCCTGTGATT |
| | | | | | TTTCAATG[T/C]AAAACA |
| | | | | | AAATTTAAAACTTCTTA |
| | | | | | TAATAGTATTTAGCAGG |
| | | | | | AGAAAAGAAGGAGGGA |
| | | | | | ACTA |
| | | | | | (SEQ ID NO: 28) |
| Quantitative | 1.13E−005 | SLC35F5, | 2 | 68429675 | ATGTTTTGCCCTTTGATA |
| | | ACTR3, LYPD1 | | | TTCACAAGGCTTCCCCA |
| | | | | | TCCTATCTTTCTGGCCTC |
| | | | | | TGATACC[T/G]TCTTACC |
| | | | | | ACCACCCTCCCTTTATTC |
| | | | | | TCTGTCCCTACTCAGCTT |
| | | | | | TATTTTTTTCCACAGGG |
| | | | | | (SEQ ID NO: 29) |
| Quantitative | 1.13E−005 | SLC35F5, | 2 | 68492186 | AAATGGCATGCTTCATG |
| | | ACTR3, LYPD1 | | | AAGACTCGGTGCACGGA |
| | | | | | CCTGATGGAATGCTGTG |
| | | | | | CCACCTCCA[T/C]TCTTA |
| | | | | | GGCCTCGCCAAGAGGCC |
| | | | | | CCCAGTTTCTAGTAGGA |
| | | | | | AACAGGTATTAATAATG |
| | | | | | CGAG |
| | | | | | (SEQ ID NO: 30) |
| Quantitative | 1.13E−005 | SLC35F5, | 2 | 68515035 | ACCAGATATTTGGTAAT |
| | | ACTR3, LYPD1 | | | TTGTTGAATGAATTAAA |
| | | | | | TGCAAATGGTTTTGAAA |
| | | | | | AAATAAAAG[A/G]TATT |
| | | | | | ATTGTTATCATTGTCTGT |
| | | | | | TGTACCTGTTTTCTTCCT |
| | | | | | TTAAAAGTCTTGGAAAA |
| | | | | | TTC |
| | | | | | (SEQ ID NO: 31) |
| Quantitative | 2.15E−006 | TPP2, ERCC5, | 12 | 76113728 | CATCCCATGGAATAAAA |
| | | KDELC1, ZWINT | | | AAGAATACAGCTTTACA |
| | | | | | CAAAACTGGTTAATGAT |
| | | | | | AACTTGCAT[A/G]TGAGC |
| | | | | | TCAGTCACTTCAGTCGT |
| | | | | | GTCCGATTCTGCGCAAC |
| | | | | | CCCATGGACTGTAGCCT |
| | | | | | GCCA |
| | | | | | (SEQ ID NO: 32) |
| Quantitative | 4.68E−006 | TPP2, ERCC5, | 12 | 76198676 | AACAAATGAATTAAGAG |
| | | KDELC1, ZWINT | | | ATATTTGGTTGCCATGA |
| | | | | | ATGTTATGTGGAAACAA |
| | | | | | GACAACTGG[T/C]GTGAT |
| | | | | | AAGGAATCTGGGAAATC |
| | | | | | TGCTTACTTTTGGTACCG |
| | | | | | AAAGCTGAGCTGACTTA |
| | | | | | ACC |
| | | | | | (SEQ ID NO: 33) |
| Quantitative | 1.21E−005 | ZWINT | 26 | 2605707 | AAGATTATACTAATTTT |
| | | | | | TGTAAAAAGGAGATTAT |
| | | | | | AGTATGATTAAACATTA |
| | | | | | ACATTCAAA[T/C]GCTGA |
| | | | | | TTTAAATTGTGCATATA |
| | | | | | TTTTTCTCATTTCTTATT |
| | | | | | GAATTTATGATCATTGA |
| | | | | | TGA |
| | | | | | (SEQ ID NO: 34) |
| Qualitative | 2.68E−005 | PPID, ETFDH, | 17 | 42419533 | CGCCCTTTTCCTGTAAC |
| (Average) | | TMEM144 | | | AAGAGGCTAGAATTTTC |
| | | | | | CACTTTTATTGCCTTGGA |
| | | | | | GAAGAAAC[A/G]AAGCA |
| | | | | | GATGTCTGCAGGAAACA |
| | | | | | AAGGAATCAAATTTGTA |
| | | | | | GTATTTAATTGCCAAAG |
| | | | | | GCCC |
| | | | | | (SEQ ID NO: 35) |
| Quantitative | 3.11E−005 | CDKN2B, IFNT | 8 | 22806810 | TCAGGGTAGGAAAGCTT |
| | | | | | TATGGTATAATTCATGC |
| | | | | | TCCAAAAGCTCCCCATA |
| | | | | | GGATCAGGT[T/G]AACC |
| | | | | | CACATCTTGGCCTGTCC |
| | | | | | TGTTGTGCCTCCCTTGTT |
| | | | | | CCATTTCAGGTTTCTCCT |
| | | | | | GAA |
| | | | | | (SEQ ID NO: 36) |
| Qualitative | 3.80E−005 | NONE | 6 | 51851114 | AAGATCTTGGCATGCTT |
| (Peak) | | | | | TCAATTCCTCAAGGAGA |
| | | | | | TAAGGAAAGAAAAATA |
| | | | | | AACAGCACTA[T/C]AGA |
| | | | | | AAAATATGAATTAGTCC |
| | | | | | TGGAAATGGCAGGTATC |
| | | | | | TCTAGGCCTTACATCAC |
| | | | | | ATTAGC |
| | | | | | (SEQ ID NO: 37) |
| Qualitative | 3.85E−005 | UBXN4, MCM6, | 2 | 64266248 | ACTACTTCAGCCTTACT |
| (Average) | | DARS, CXCR4 | | | CTTTTAGAATTGTAGTC |
| | | | | | AGAAAAGATTGTGAGTC |
| | | | | | GTTTGGAAA[T/C]GAGC |
| | | | | | ACTTAGCCCATTTCTATC |
| | | | | | GCACGCTGGAAACTATG |
| | | | | | AACATTTTCACTGCACG |
| | | | | | TACA |
| | | | | | (SEQ ID NO: 38) |
| Qualitative | 3.87E−005 | NONE | 6 | 51772055 | CTGCTTGGAAAATTCAT |
| (Peak) | | | | | ACCAAAAGCAGTAACA |
| | | | | | GGAAATGTGCAGAGGGT |
| | | | | | TTTCTGTGTG[T/C]CCAG |
| | | | | | CACAGTGTAAGTAAGCT |
| | | | | | TAATAATCCCTATTTTCT |
| | | | | | TAATTCATTCCACACAA |
| | | | | | AGGA |
| | | | | | (SEQ ID NO: 39) |
| Quantitative | 4.02E−005 | STAC, LRRFIP2, | 22 | 10999946 | CAAAGAGGTCTGAGTTT |
| | | PLCD1 | | | CTGATGGCAATTCATAA |
| | | | | | AGTGACACTGAATTCCA |
| | | | | | AGAGTAAAT[A/G]TCTG |
| | | | | | AAAAAAGCCAGAGCCA |
| | | | | | TCTAAATAGAACAGCAG |
| | | | | | AGAGAGGAGGACAGAG |
| | | | | | AAAGCAG |
| | | | | | (SEQ ID NO: 40) |
| Qualitative | 4.49E−005 | COMMD2, PFN2, | 1 | 120575773 | CAGGCCACCTGAATTGG |
| (Average) | | RNF13, | | | ATCTTTGCTTCTCTACTT |
| | | TIM4SF4, | | | TTTAGTAGTAACTTTGG |
| | | TIM4SF1, | | | TAAGTTAC[A/G]TAACCT |
| | | TM4SF18, GYG1, | | | CCCAGGGCCTCAGATTT |
| | | CPB1, AGTR1 | | | CTCACCTGTAAAGTGGG |
| | | | | | AGTAATGTGCACACCTG |
| | | | | | GTA |
| | | | | | (SEQ ID NO: 41) |
| Qualitative | 4.54E−005 | R3HDM2 | 16 | 8218820 | AAATGGCTTTTTTACAT |
| (Average) | | | | | GAAGCAACATGTACCAG |
| | | | | | GACCTCTATTATTCACT |
| | | | | | ACAAAGAAA[A/G]TAGT |
| | | | | | GAATAATGCTCCATGTC |
| | | | | | TCAGAATTCTTGTGTAT |
| | | | | | AAAATGAGAATTATCAA |
| | | | | | ATACT |
| | | | | | (SEQ ID NO: 42) |
| Quantitative | 4.72E−005 | ARPP-19, CYP19, | 10 | 59551460 | ATAGGGAGAGAGTTATA |
| | | SCG3, LYSMD2, | | | CTCCTGCAAAGGACTGT |
| | | TMOD3, LEO1, | | | TTACCCCCCTTCAAAAA |
| | | GNB5 | | | TGTGCATTA[A/G]TCATT |
| | | | | | AGCTAAGTGACAGTGGG |
| | | | | | TAGATGGAAAGGTGACT |
| | | | | | TTATTCAGTGATATTTTT |
| | | | | | CTA |
| | | | | | (SEQ ID NO: 43) |
| Qualitative | 4.76E−005 | DDX10, FDX1, | 15 | 18460127 | CTTATACATAAAGTTCA |
| (Peak) | | RDX | | | ATTTTTTTTATATGGTAT |
| | | | | | AAAATGCCCTTTAAGAT |
| | | | | | CTGGCTCA[A/G]TGTCTT |
| | | | | | CTTATCAACCTCATTTTT |
| | | | | | CACTTCCCATTTTTTTCA |
| | | | | | GTTCTGCACACAGTCCC |
| | | | | | A |
| | | | | | (SEQ ID NO: 44) |

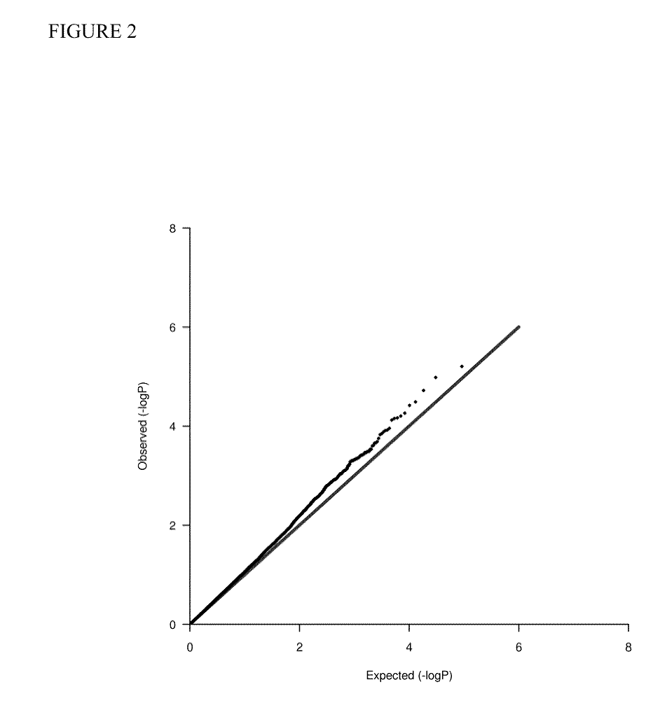

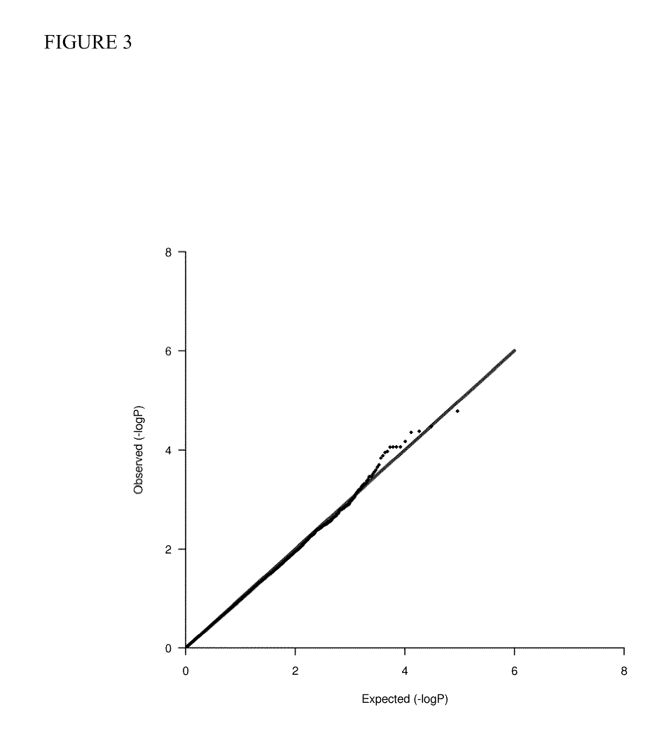

Association of loci with Map fecal positive (fecal positive vs. fecal negative). Single SNP analysis was conducted to test the association of loci with a positive Map fecal culture. Cases in this analysis were defined as animals with a positive fecal culture result (n=41) and controls were defined as animals with a negative fecal culture result (n=168), regardless of culture of tissue status. The standard allelic, dominance, recessive, and genotypic models showed evidence that the results were influenced by population substructure (identified by Q-Q-plot, FIG. 2 and genomic inflation factor, λ_{gc}=1.10). This was likely due to Herds A, B, and C containing fewer fecal positive animals relative to Herd D. To account for population substructure, a stratified analysis (CMH test) was performed within each herd. Investigation of the resulting Q-Q-plot (FIG. 3) and the genomic inflation factor (λ_{gc}=1.0) shows this test successfully accounted for population substructure. CMH test results showed no strong associations with a P value of less than 5×10⁻⁷; however, four SNPs were found to be moderately significant (p<5×10⁻⁵) located on chromosomes 5, 8, 9, and 23 (Table 1 above).

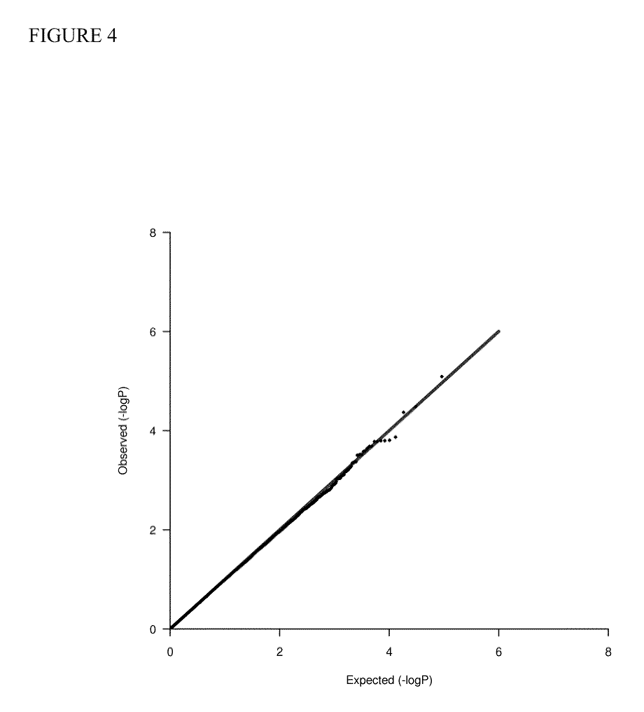

Association of loci with Map tissue infection but fecal culture negative (infected). Single SNP analysis was conducted to test the association of loci with Map tissue infection but with a negative fecal culture result. Cases in this analysis were defined as animals with a positive culture of tissue result and a negative fecal culture (n=54), and controls were defined as animals with a negative culture of tissue result and a negative fecal culture (n=112). No strong association with any SNPs was identified using the allelic, dominant, recessive, or genotypic model, but three SNPs were identified with moderate P values using the allelic model. Two SNPS located on chromosomes 3 and 21 were the same as those found associated with tissue positive status. The third SNP was a newly identified locus found on chromosome 1, 14 Mb downstream of the SNPs found with a moderate association with a tissue positive result (Table 3A). Analysis of “infected” animals did not show evidence for population substructure in the Q-Q-plot (FIG. 4) or genomic inflation factor (λ_{gc}=1).

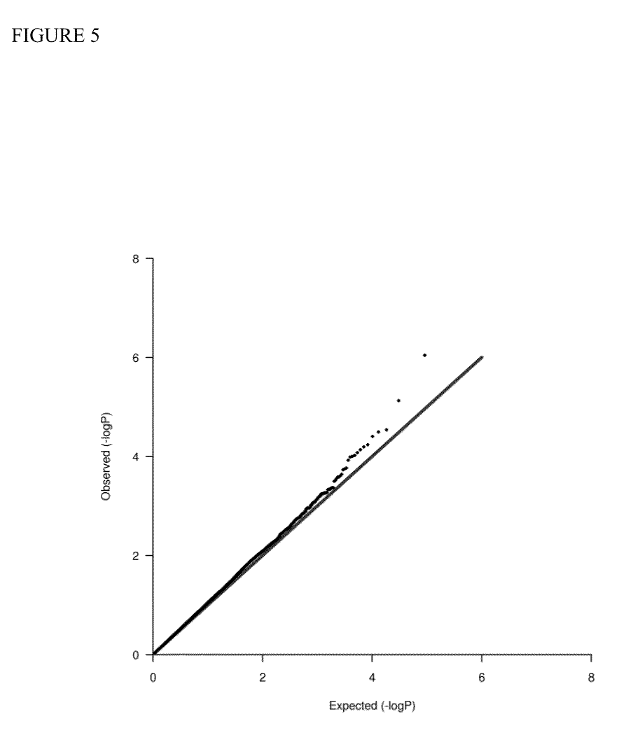

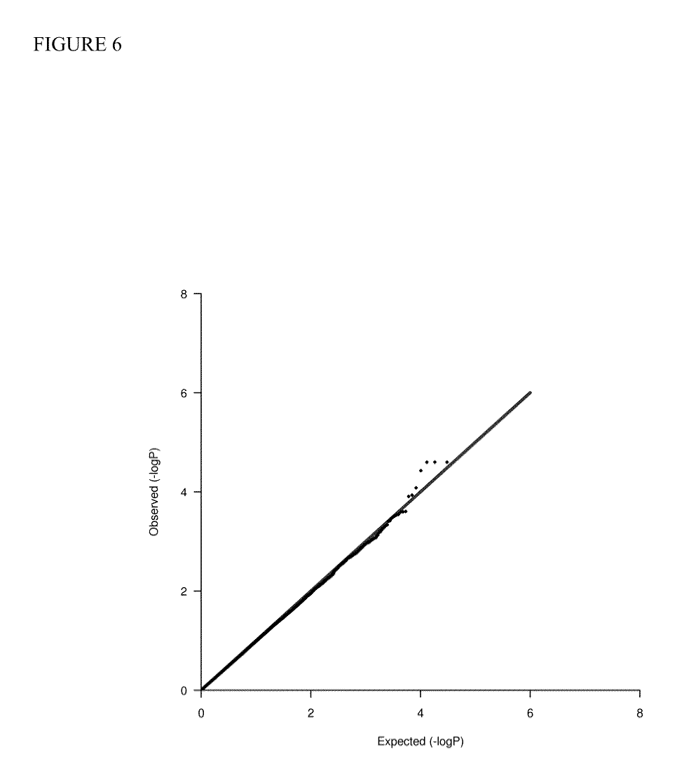

Association of loci with the class Map tissue infected, fecal positive (clinical). Single SNP analysis was conducted to test the association of loci with a Map tissue infection and positive fecal culture (“clinical”). Cases in this analysis were defined as animals with a positive tissue and a positive fecal culture result (n=25), while controls were defined as animals with a negative tissue and a negative fecal culture result (n=112). The standard allelic, dominance, recessive, and genotypic models showed evidence that results were influenced by population substructure (identified by Q-Q-plot, FIG. 5 and genomic inflation factor, λ_{gc}=1.06). To account for population substructure a stratified analysis (CMH test) was performed within the herds. Investigation of the resulting Q-Q-plot (FIG. 6) and the genomic inflation factor (GC=1.0) shows this test successfully accounted for population substructure. One strong association was found with SNP BTB-01957421 located on BTA 9 using the CMH test stratifying within herd (p=1×10⁻⁷; P=0.004 after Bonferroni correction). Four other SNPs, 3 located on chromosome 9 adjacent to BTB-01957421, showed moderate association (p=2.5×10⁻⁵) as did one on chromosome 7 (p=3.72×10⁻⁵) (Table 3A).

### REFERENCES CITED FOR THIS EXAMPLE 1, AND INCORPORATED HEREIN FOR THEIR RESPECTIVE TEACHINGS

1. USDA—Animal and Plant Health Inspection Service (APHIS) (2008), Johne's Disease in U.S. Dairies, 1991-2007. USDA Info Sheet, April.
2. Bentley R. W., Keenan J. I., Gearry R. B., Kennedy M. A., Barclay M. L. & Roberts R. L. (2008) Incidence of Mycobacterium avium subspecies paratuberculosis in a population-based cohort of patients with Crohn's disease and control subjects. American Journal of Gastroenterology 103(5) 1168-1172.
3. Clarke C. J. (1997) The pathology and pathogenesis of paratuberculosis in ruminants and other species. Journal of Comparative Pathology 116, 217-261.
4. Collins M. T., Gardner I. A., Garry F. B., Roussel A. J. & Wells S. J. (2006) Consensus recommendations on diagnostic testing for the detection of paratuberculosis in cattle in the United States. Journal of the American Veterinary Medical Association 229, 1912-1919.
5. Gonda M. G., Chang Y. M., Shook G. E., Collins M. T. & Kirkpatrick B. W. (2006) Genetic variation of Mycobacterium avium ssp paratuberculosis infection in US Holsteins. Journal of Dairy Science 89, 1804-12.
6. Gonda M. G., Kirkpatrick B. W., Shook G. E. & Collins M. T. (2007) Identification of a QTL on BTA20 affecting susceptibility to Mycobacterium avium ssp. paratuberculosis infection in US Holsteins. Animal Genetics 38, 389-396.
7. Harris N. B. & Barletta R. G. (2001) Mycobacterium avium subsp. paratuberculosis in veterinary medicine. Clinical Microbiology Review 14, 489-512.
8. Hinger M., Brandt H., Horner S. & Erhardt G. (2007) Short Communication: Association Analysis of Microsatellites and Mycobacterium avium Subspecies paratuberculosis Antibody Response in German Holsteins. Journal of Dairy Science 90, 1957-1961.
9. Hinger M., Brandt H. & Erhardt G. (2008) Heritability estimates for antibody response to Mycobacterium avium subspecies paratuberculosis in German Holstein cattle. Journal of Dairy Science 91, 3237-3244.
10. Koets A. P., Adugna G., Janss L. L. G., van Weering H. J., Kalis C. H. J., Wentink G. H., Rutten V. P. M. G. & Schukken Y. H. (2000) Genetic Variation of Susceptibility to Mycobacterium avium subsp. paratuberculosis Infection in Dairy Cattle. Journal of Dairy Science 83, 2702-2708.
11. Matukumalli L. K., Lawley C. T., Schnabel R. D., Taylor J. F., Allan M., Heaton M. P., O'Connell J. R., Sonstegard T. S., Smith T. P. L., Moore S. S. & Van Tassell C. P. (2008) Development and characterization of a high density SNP genotyping assay for cattle. Genome Res. (Submitted).
12. McCartney S. A., Ballinger A. B., Vojnovic I., Farthing M. J. G. & Warner T. D. (2002) Endothelin in human inflammatory bowel disease: comparison to rat trinitrobenzenesulphonic acid-induced colitis. Life Sciences 71, 1893-1904.
13. McKenna S. L. B., Keefe G. P., Barkema H. W. & Sockett D. C. (2005) Evaluation of three ELISAs for Mycobacterium avium subsp. paratuberculosis using tissue and fecal culture as comparison standards. Veterinary Microbiology 110, 105-111.
14. Momotani E., Whipple D. L., Thiermann A. B. & Cheville N. F. (1988) Role of M cells and macrophages in the entrance of Mycobacterium paratuberculosis into domes of ileal peyer's patches in calves. Veterinary Pathology 25, 131-137.
15. Mortensen H., Nielsen S. S. & Berg P. (2004) Genetic Variation and Heritability of the Antibody Response to Mycobacterium avium subspecies paratuberculosis in Danish Holstein Cows. Journal of Dairy Science 87, 2108-2113.
16. Ott S. L., Wells S. J. & Wagner B. A. (1999) Herd-level economic losses associated with Johne's disease on US dairy operations. Preventative Veterinary Medicine 40, 179-192.
17. Purcell S., Neale B., Todd-Brown K., Thomas L., Ferreira M. A. R., Bender D., Maller J., Sklar P., de Bakker P. I. W., Daly M. J. & Sham P. C. (2007) PLINK: a toolset for whole-genome association and population-bases linkage analysis. American Journal of Human Genetics 81, 559-575.
18. Reddacliff, L. A., Beh K., McGregor H. & Whittington R. J. (2005) A preliminary study of possible genetic influences on the susceptibility of sheep to Johne's disease. Australian Veterinary Journal 83, 435-441.
19. Takizawa S., Uchide T., Adur J., Kozakai T., Kotake-Nara E., Quan J. & Saida K. (2005) Differential expression of endothelin-2 along the mouse intestinal tract. Journal of Molecular Endocrinology 35, 201-209.
20. Taylor K. H., Taylor J. F., White S. N. & Womack J. E. (2006) Identification of genetic variation and putative regulatory regions in bovine CARD15. Mammalian Genome 17, 892-901.
21. Waters W. R., Stabel J. R., Sacco R. E., Harp J. A., Pesch B. A. & Wannemuehler M. J. (1999) Antigen-specific B-cell unresponsiveness induced by chronic Mycobacterium avium subsp. paratuberculosis infection of cattle. Infection and Immunity 67, 1593-1598.
22. Wellcome Trust Case Control Consortium (2007) Genome-wide association study of 14,000 cases of seven common diseases and 3,000 shared controls. Nature 447, 661-78.
23. Whitlock R. H., Wells S. J., Sweeney R. W. & Van Tiem J. (2000) ELISA and fecal culture for paratuberculosis (Johne's disease): sensitivity and specificity of each method, Veterinary Microbiology 77, 387-398.
24. Whitlock R. H., Rosenberger A. E., Sweeney R. W. & Spencer P. A. (1996) Distribution of M. paratuberculosis in tissues of cattle from herds infected with Johne's disease. In: Proceedings of the Fifth International Colloquium on Paratuberculosis (ed. By R. J. Chiodini, M. E. Hines & M. T. Collins), 29th September-4 Oct. 1996, International Association for Paratuberculosis, 44 Francis Street, Rehoboth, Mass., USA, 168-174.
25. Whittington R. J. & Sergeant E. S. G. (2001) Progress towards understanding the spread, detection and control of Mycobacterium avium subsp. paratuberculosis in animal populations. Australian Veterinary Journal 79, 267-278.
26. Wu C.-W., Livesey M., Schmoller S. K., Manning E. J. B., Steinberg H., Davis W. C., Hamilton M. J. & Talaat A. M. (2007) Invasion and persistence of Mycobacterium avium subsp. paratuberculosis during early stages of Johne's disease in calves. Infection and Immunity 75, 2110-2119.
27. Devlin B. & Roeder K. (1999) Genomic control for association studies. Biometrics 55, 997-1004.

### Example 2

### Identification of Loci Associated with Tolerance to Johne's Disease in U.S. Holstein Cattle

Overview. Johne's disease is an incurable contagious bacterial illness caused by Mycobacterium avium subspecies paratuberculosis (Map). While the heritability of tolerance has been estimated at 0.09±0.036, the loci associated with tolerance have not yet been investigated. The low sensitivity of the current diagnostic techniques and the long incubation period (4 to 5 years) until the appearance of clinical signs are some of the major road blocks to controlling the disease (Chiodini & Merkal 1984; Collins et al. 2006).

The eradication of the disease has not been successfully obtained by traditional methods, and therefore new approaches are needed. One such approach is the genetic selection of animals that are tolerant to Johne's disease. Cattle have evolved defense mechanisms to fight pathogens, such as resistance and tolerance. Co-evolution between hosts and pathogens is believed to increase the biological diversity among animals and pathogens (Burdon and Muller 1987). Tolerance aims to reduce the harm caused by the parasite, whereas resistance prevents the infection of the pathogen. For the pathogen, tolerance results in a reduction of virulence, thereby increasing selection pressure for pathogens to exhibit higher growth rates. For the host, tolerance will tend to increase disease prevalence, while reducing the individual risk of death from the disease (Miller et al. 2006). Weiss and colleagues (2006) suggested that a state of tolerance may exist in the intestine of cows sub-clinically infected with Mycobacterium avium subsp. paratuberculosis after they analyzed the mucosal immune response of these cows. Zanella and colleagues (2008), found that tolerance to bovine paratuberculosis is heritable with estimates of 0.09±0.036. Gonda et al. (2007) were the first to report evidence of QTL (Quantitative Trait Loci) on chromosome 20 (BTA20) associated with susceptibility to Johne's disease. However the investigation or identification of the loci associated with tolerance to animal disease has not been undertaken.

The purpose of this EXAMPLE 2 was to identify loci associated with tolerance to Johne's disease in cows infected with Map using a genome-wide approach. Such identification could be used to develop a marker-assisted selection program to reduce the severity and the losses caused by Johne's disease in cattle.

Feces, ileum, two mesenteric lymph nodes, and tissue from the ileo-cecal valve were harvested and cultured for Map from 260 Holstein cows from four dairy herds. Ninety-four cows had a Map tissue colony-forming unit (CFU) value≧1; 42 of these had fecal CFU values≧1, and 8 animals had fecal CFU values exceeding their tissue CFU values. To compute a tolerance (T) index, the fecal CFU value+100 was divided by the tissue CFU value+100. Peak and average fecal and tissue values were used to determine Tₚₑₐₖ and T_{average} for each animal. Genotyping of these 94 animals was conducted with the Illumina bovineSNP50 bead array. After quality filtering and genotype pruning, 45,591 SNPs for 89 animals remained. The results did not show evidence for population substructure (genomic inflation factor, based on median Chi-sq statistic λ_{GC}=1.03). Whole genome association analysis was conducted using the R statistical environment and PLINK. Tolerance values were treated as a quantitative trait and compared with allele frequencies for each SNP. Strong evidence for association was identified with Tₚₑₐₖ and a locus on BTA15 (P=1.8×10⁻⁷, after Bonferroni correction P=0.0079) while moderate evidence for association (P=3.8×10⁻⁵) was identified on two adjacent SNPs on BTA 6. The same SNP on BTA 15 showed moderate evidence for association (P=3×10⁻⁶) with T_{average}. Four additional SNPs also showed moderate evidence for association on BTA 17 (P=2.68×10⁻⁵), BTA 2 (P=3.8×10⁻⁵), BTA 1 (P=4.5×10⁻⁵) and BTA 16 (4.5×10⁻⁵). This is the first study to evaluate and identify genetic tolerance loci with disease in cattle.

### Materials & Methods:

Selection of Animals and Data Collection. Fecal samples for two hundred fifty-four Holstein cows from four dairy herds located in New York (Herd A), Pennsylvania (Herd B and Herd C), and Vermont (Herd D) were cultured every 3 to 6 months between January 1999 and November 2007 or until the animals were culled. The disease status of the animals was determined by the number of colony forming units of Map from culture of tissue samples of the ileum, ileo-cecal valve, and two adjacent ileo-cecal lymph nodes harvested at slaughter, as described by Whitlock et al. (1996 and 2000).

Tolerance was estimated in animals that in which the tissue was infected with Map. The disease status of the animals was determined by the results of the tissue culture, because of the higher sensitivity of tissue culture compared to fecal culture or ELISA. Animals were considered to be Johne's positive if they had one or more colony-forming units per gram of tissue (CFUₜ/g>1) in any of the four tissues examined. Ninety-four (36%) of the animals tested had tissue infected with Map (Table 4). The mean age of animals was 59 months, with a range of 22.87 to 135 months.

TABLE 4
Distribution of infected animals by herd.

| Herd | Total |
|---|---|
| Herd A | 30 |
| Herd B | 9 |
| Herd C | 16 |
| Herd W | 39 |
| Total | 94 |

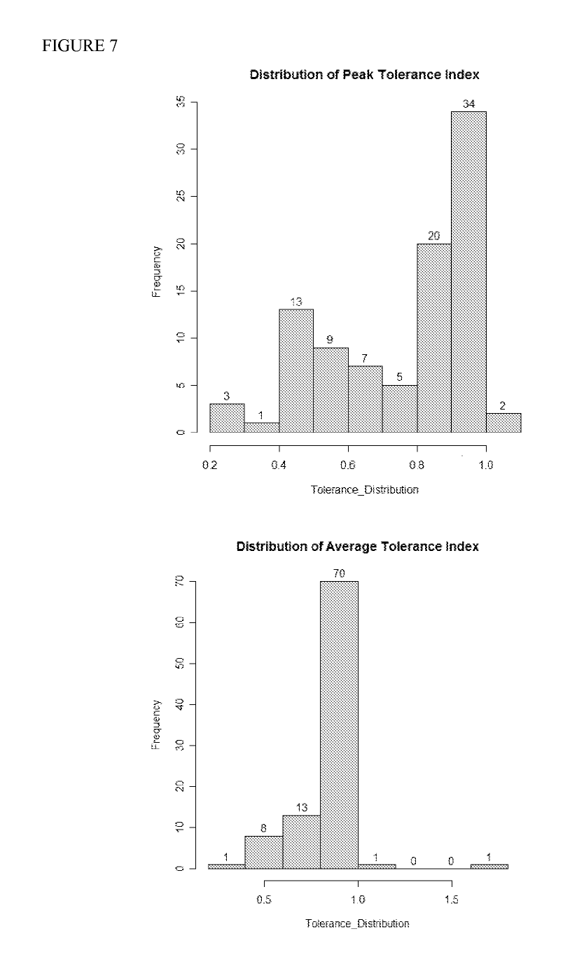

The tolerance index (T) was calculated using the peak (T_{Peak}) and average (T_{Average}) CFUs for feces and tissue taken at slaughter. The tolerance index was defined as the fecal CFUs (CFU_{f})+100 divided by the tissue CFUs (CFUₜ)+100 (FIG. 7).

DNA Preparation and Genotyping. DNA was extracted from 15-40 mg of tissue from each animal using the Puregene DNA extraction kit per manufacturer's instructions (Gentra, Minneapolis, Minn.). DNA samples were quantified using NanoDrop spectrophotometry, and DNA purity was estimated using the 260/280 ratio. Samples with 260/280 ratios below 1.8 and higher than 2.0 were excluded because of possible protein and (or) RNA contamination that could compromise the quality of the genotype. Five micrograms of DNA were diluted to a final concentration of 50 ng/μl and genotyped with the Illumina BovineSNP50 BeadChip as described (Matukumalli et al., 2008). The Illumina BovineSNP50 beadchip assay contains 55,074 SNPs with a mean spacing of one SNP every 35 kb across the bovine genome.

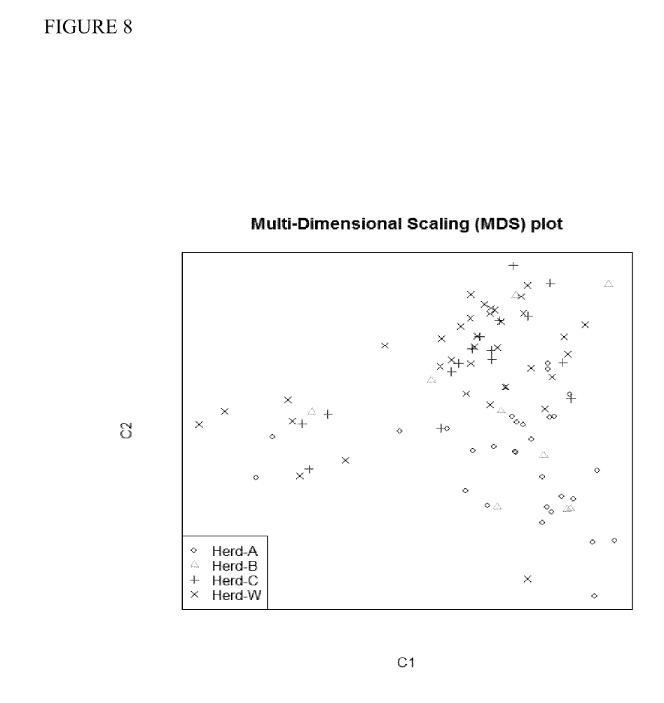

Quality Assurance. All samples were brought into a single BeadStudio file, and genotypes were identified using a custom cluster file that was based on >2,000 samples from multiple cattle breeds. Samples were first evaluated for quality control. Two samples were removed from the analysis because of a no call rate greater than 10%. These samples were removed because samples with a call-rate less than 90% may be due to degraded DNA. Multidimensional scaling analysis (MDS) plotted the genome-wide identity-by-state (IBS) of the SNPs to identify animals with significant genetic variation. Animals (n=3) with different genetic backgrounds were removed from the analysis. After exclusion of the outlier from the analyses, MDS plot showed all the animals with similar genetic ancestry (FIG. 8). After the removal of animals for quality control, eighty-nine animals (n=89) remained for the analyses with a call rate of 98.9%. In addition to evaluation of the samples for quality, the SNPs were also scrutinized. Monomorphic SNPs (6,356), SNPs with minor allele frequencies less than 1% (8,185), and SNPs with greater than a 10% no call-rate (1,503) were removed from the analysis. After frequency and genotyping pruning, 45,591 SNPs were left for the association analysis.

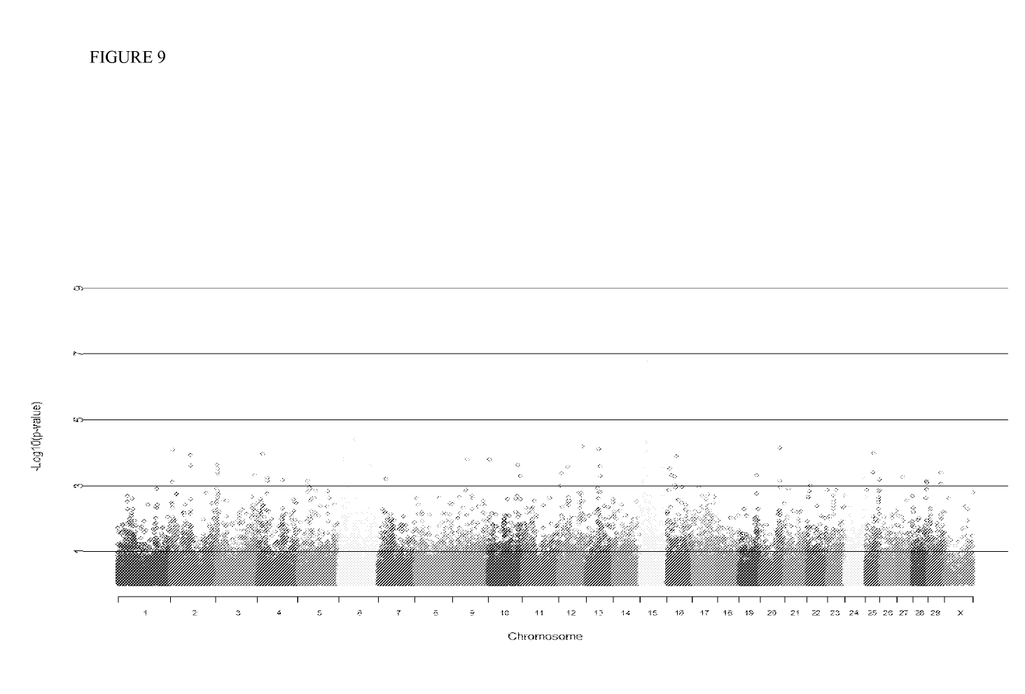

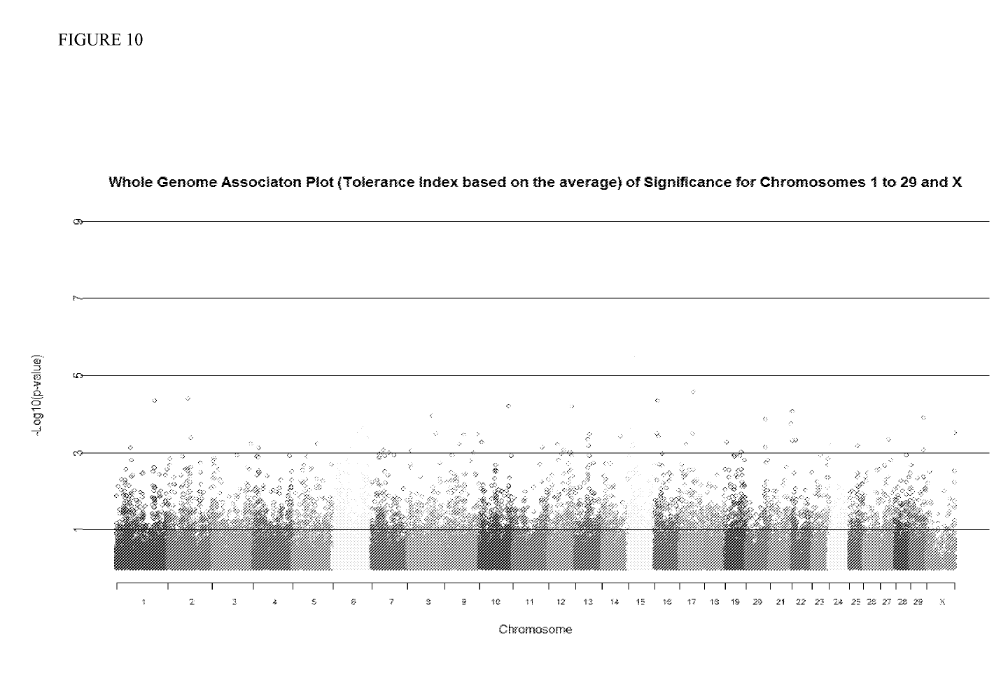

Association Analysis. The whole genome association study was conducted to find SNP's associated with the spectrum of tolerance values in animals with tissue infected with Map (FIGS. 9 and 10). For this approach tolerance was treated as a quantitative trait. T_{Peak} values ranged from T_{Peak}=0.27 to 1.07 and T_{Average} value ranged from T_{Average}=0.35 to 1.64. The whole genome association analysis was conducted using the R statistical environment and PLINK (Purcell et al 2007, Version 1.04). The likelihood ratio test and the Wald statistical test were used within PLINK. Significance for association tests were based on the recommendation of the Welcome Trust Case Control Consortium (2007) where unadjusted P values less than 5×10⁻⁷ were considered to provide strong evidence of association and unadjusted P values between 5×10⁻⁵ and 5×10⁻⁷ were considered to provide moderate evidence for association. Associations with strong evidence as defined by the Welcome Trust Case Control Consortium coincide with the associations identified after Bonferroni correction for multiple testing.

### Results:

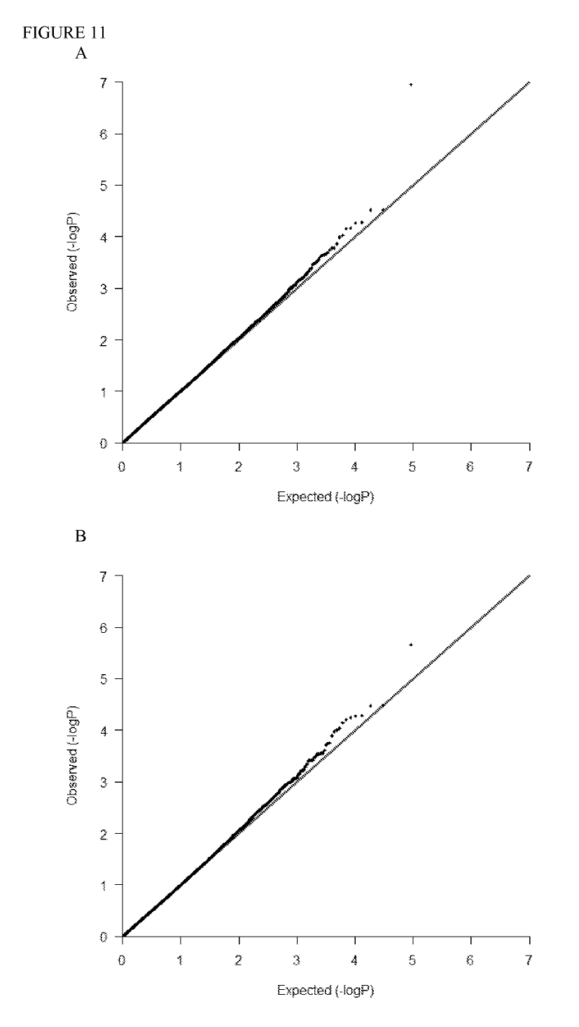

Quantitative analysis for association of loci with tolerance. Each SNP was analyzed for the presence of an association with peak and average tolerance. Multidimensional scaling plot, QQ-plot, and the genomic inflation factor were evaluated to identify population stratification using the quantitative measure of tolerance of T_{Peak}. No evidence for population stratification was observed (FIG. 11A). Strong evidence for association for T_{Peak} was found with SNP BTB-00584953, located on BTA 15, at position 21,254,062 by using the basic allelic model (unadjusted P=1.8×10⁻⁷; P=0.0079 after Bonferroni correction). A second SNP on BTA 15, located 2.8 Mb from BTB-00584953, was associated (P=4.7×10⁻⁵) with T_{Peak}. two other SNPs, located 79 Kb apart from each other on BTA 6, were found to have a moderate significance using the basic allelic model (unadjusted P=3.8×10⁻⁵). Similar to the quantitative measure of tolerance T_{Peak}, T_{Average} did not show evidence for population stratification after analysis with the multidimensional scaling plot, QQ plots or the genomic inflation factor (FIG. 11B). Using the basic alleleic model, moderate evidence for association (P=3.035×10⁻⁶) for with T_{Average} was shown with the same SNP on BTA 15 as was identified with T_{Peak}. Additional SNPs located on BTA 17 (unadjusted P=2.68×10⁻⁵), BTA 2 (unadjusted P=3.8×10⁻⁵), BTA 1 (unadjusted P=4.5×10⁻⁵) and BTA 16 (unadjusted P=4.5×10⁻⁵) also showed moderate evidence for association with T_{Average}.

### REFERENCES CITED FOR THIS EXAMPLE 2, AND INCORPORATED HEREIN FOR THEIR RESPECTIVE TEACHINGS

1. APHIS Johne's Disease on U.S. Diaries 1991-2007 Animal and Plant Health Inspection Service (APHIS)., 2008, p 3.
2. Abubakar, I.; Myhill, D.; Aliyu, S. H. & Hunter, P. R. Detection of Mycobacterium avium subspecies paratuberculosis from patients with Crohn's disease using nucleic acid-based techniques: a systematic review and meta-analysis. Inflamm Bowel Dis, 2008, 14, 401-410
3. Alaniz, R. C.; Thomas, S. A.; Perez-Melgosa, M.; Mueller, K.; Fan, A. G.; Palmiter, R. D. & Wilson, C. B. Dopamine beta-hydroxylase deficiency impairs cellular immunity. Proc Natl Acad Sci, 1999, 96, 2274-2278
4. Banerjee, S. & Bond, J. S. Prointerleukin-18 is activated by meprin beta in vitro and in vivo in intestinal inflammation. J Biol Chem., 2008
5. Burdon J J, M. W Measuring the cost of resistance to Puccinia coronata Cda in Avena fatua L. Journal of Applied Ecology, 1987, 24, 191-200.
6. Chiodini, R. J., V. K. H. & Merkal, R. Ruminant paratuberculosis (Johne's disease): the current status and future prospects. Ruminant paratuberculosis (Johne's disease): the current status and future prospects. Cornell Vet., 1984, 74, 218-62.
7. Churchill G A, Doerge R W. Empirical threshold values for quantitative trait mapping. Genetics. 1994; 138:963-71.
8. Collins, M. T.; Gardner, I. A.; Garry, F. B.; Roussel, A. J. & Wells, S. J. Consensus recommendations on diagnostic testing for the detection of paratuberculosis in cattle in the United States. J Am Vet Med Assoc, 2006, 229, 1912-1919
9. Gonda, M. G.; Chang, Y. M.; Shook, G. E.; Collins, M. T. & Kirkpatrick, B. W. Effect of Mycobacterium paratuberculosis infection on production, reproduction, and health traits in US Holsteins. Prev Vet Med, 2007, 80, 103-119
10. Gonda, M. G.; Kirkpatrick, B. W.; Shook, G. E. & Collins, M. T. Identification of a QTL on BTA20 affecting susceptibility to Mycobacterium avium ssp. paratuberculosis infection in US Holsteins. Anim Genet, Dairy Science, 2007, 38, 389-396
11. Harris, N. B. & Barletta, R. G. Mycobacterium avium subsp. paratuberculosis in Veterinary Medicine. Clin Microbiol Rev. 2001, 14, 489-512
12. Imtiaz F, Savilahti E, Sarnesto A, Trabzuni D, A l-Kahtani K, Kagevi I, Rashed M S, Meyer B F, JärveläI. The T/G 13915 variant upstream of the lactase gene (LCT) is the founder allele of lactase persistence in an urban Saudi population. J Med Genet, 2007 oct, 44(10):e89.
13. Li, J.; Zhou, Y. & Elston, R. C. Haplotype-based quantitative trait mapping using a clustering algorithm. BMC Bioinformatics, 2006, 7, 258
14. Li, J.; Yu, L.; Shen, Y.; Zhou, L.; Wang, Y. & Zhang, J Inhibition of CXCR4 activity with AMD3100 decreases invasion of human colorectal cancer cells in vitro. World J Gastroenterol, 2008, 14, 2308-2313
15. Matukumalli, L. K.; Lawley, C. T.; Schnabel, R. D.; Taylor, J. F.; Allan, M.; Heaton, M. P.; O'Connell, J. R.; Sonstegard, T. S; Smith, T. P. L.; Moore, S. S. and Van Tassell, C. P. Development and characterization of a high density SNP genotyping assay for cattle. 2008. (Submitted)
16. Miller, M. R.; White, A. & Boots, M. The evolution of parasites in response to tolerance in their hosts: the good, the bad, and apparent commensalism. Evolution, 2006, 60, 945-956.
17. Nordlund, K. V.; Goodger, W. J.; Pelletier, J. & Collins, M. T. Associations between subclinical paratuberculosis and milk production, milk components, and somatic cell counts in dairy herds. J Am Vet Med Assoc, Department of Medical Sciences, 1996, 208, 1872-1876
18. Ott, S. L.; Wells, S. J. & Wagner, B. A. Herd-level economic losses associated with Johne's disease on US dairy operations. Prev Vet Med, 1999, 40, 179-192
19. Purcell S, Neale B, Todd-Brown K, Thomas L, Ferreira MAR, Bender D, Maller J, Sklar P, de Bakker P I W, Daly M J & Sham P C (2007) PLINK: a toolset for whole-genome association and population-bases linkage analysis. American Journal of Human Genetics, 81.
20. Smyth, R. H., C. G. & Smyth, R. H., C. G. Some observations on Johne's disease with a further note on the examination of fecal samples. Vet Ret, 1950, 62, 429-450
21. Sockett, D. C.; Can, D. J. & Collins, M. T. Evaluation of conventional and radiometric fecal culture and a commercial DNA probe for diagnosis of Mycobacterium paratuberculosis infections in cattle. Can J Vet Res. 1992, 56, 148-153
22. Thony, B.; Blau, N.: Mutations in the GTP cyclohydrolase I and 6-pyruvoyl-tetrahydropterin synthase genes. Hum. Mutat. 1997, 10: 11-20.
23. Xu, J.; Zhang, S.; You, C.; Huang, S.; Cai, B. & Wang, X. Expression of human MCM6 and DNA Topo II alpha in craniopharyngiomas and its correlation with recurrence of the tumor. J Neurooncol, 2007, 83, 183-189.
24. Weiss, D. J.; Evanson, O. A. & Souza, C. D. Mucosal immune response in cattle with subclinical Johne's disease. Vet Pathol, Department of Veterinary, 2006, 43, 127-135 Wellcome Trust Case Control Consortium. Genome-wide association study of 14,000 cases of seven common diseases and 3,000 shared controls. Nature 2007, 447:661-78
25. Whitlock, R. H.; Wells, S. J.; Sweeney, R. W. & Tiem, J. V. ELISA and fecal culture for paratuberculosis (Johne's disease): sensitivity and specificity of each method. Vet Microbiol, 2000, 77, 387-398
26. Whitlock, R. H., Rosenberger, A. E., Sweeney, R. W., Spencer, P. A., Distribution of M. paratuberculosis in tissues of cattle from herds infected with Johne's disease. Proceedings of the Fifth International Colloquium on Paratuberculosis, 1996, 4:168-174.
27. Zanella, R., Settles, M., Fyock, T., Whitlock, R., Schukken, Y., Van Kessel, J., Karns, J., Hoving, E., Smith, J., Van Tassel, C., Gaskins, C. and Neibergs, H. Heritability of Genetic Tolerance to Johne's Disease Poster ASAS, Indianapolis., 2008.

### Example 3

### Identification of Map Infection Linkage Disequilibrium Region, and Loci Associated with Tolerance to Johne's Disease in U.S. Holstein Cattle

Overview. Applicants have herein identified loci that are associated with tissue infection of cattle with Map (see also Applicants' Settles et al, doi:10.111/j.1365-2052.2009.01896.x publication; incorporated herein in its entirety for its teaches on these loci, mutants thereof, and diagnostic uses thereof).

### Materials and Methods:

The population of animals used in the infection study was 245 Holstein cows from dairies in New York, Pennsylvania and Vermont that were followed to culling. Culling may have occurred for any reason, including having a Map positive diagnostic test. Fecal, ileum, ileo-cecal valve and two ileo-cecal lymph nodes were assessed for the presence of Map in each culled cow. An animal was considered tissue infected if a sample contained at least one colony-forming unit per gram of tissue (cfu/g). Fecal samples were also considered positive for Map if one or more cfu/g were detected. Each animal was genotyped with the Illumina BovineSNP50 BeadChip and after quality assurance filtering, 218 animals and 45,683 SNPs remained.

A case-control genome wide association study was conducted to test four different classifications of Map infection status (Cases) when compared to a Map negative control group (Control): presence of Map in the tissue, presence of Map in feces, presence of Map in both tissue and feces and presence of Map in tissue but not feces

### Results:

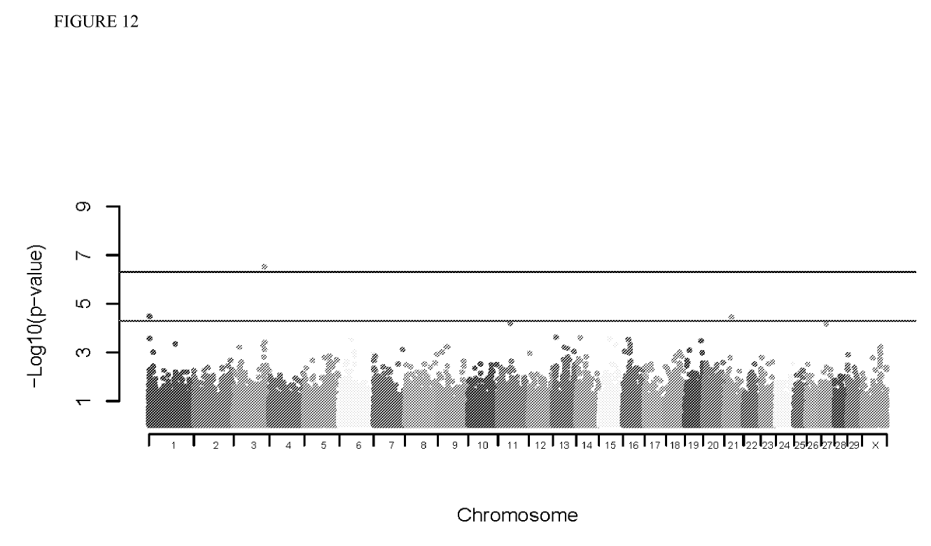

Regions on chromosomes 1, 5, 7, 8, 16, 21 and 23 were identified that showed moderate significance (P<5×10⁻⁵). A region on chromosome 3 (FIG. 12) was identified with a high level of association to the presence of Map in tissue (P=3×10⁻⁷, genome-wide Bonferonni correction for multiple testing P<0.05).

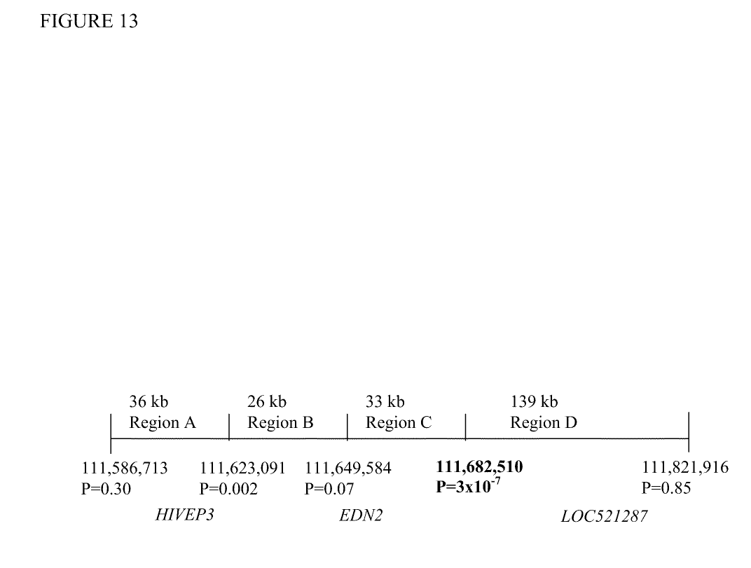

A 235 kb region (SEQ ID NO:45) associated with Map tissue infection on chromosome 3 was defined by 3 genetic markers (single nucleotide polymorphisms or SNPs) where the evidence of association fell away in both directions (FIG. 13).

FIG. 2 shows locations of single nucleotide polymorphisms (SNPs) (in base pairs) on bovine chromosome 3. The SNP at 111,682,510 (SS86341066) was the most strongly associated with tissue infection (P=3×10⁻⁷).

In particular preferred aspects, the 235 kb region (SEQ ID NO:45), or a 255,586 by region (SEQ ID NO:1) of bovine chromosome 3, referred to herein at the inventive “Map infection linkage disequilibrium region” is disclosed to be associated with Map tissue infection (P=7×10⁻⁸), and is further characterized as including three functionally relevant genes: HIVEP3 (Human immunodeficiency virus type I enhancer-binding protein 3) (SEQ ID NO:2); EDN2 (Endothelin 2) (SEQ ID NO:3); and LOC521287 (Bos taurus similar to forkhead box O6) (SEQ ID NO:4), and the respective coding transcripts (SEQ ID NOS:5, 6 and 7, respectively), and polypeptides (SEQ ID NOS:8, 9 and 10, respectively).

Endothelin 2 (EDN2) (SEQ ID NO:3), one of only three genes in this region, resides within 37 kb of the most highly associated SNP on chromosome 3. This gene is known to have a physiological function in Crohn's disease in humans, is a potent vasoconstrictor and is a positive regulator of chemotaxis in macrophages in response to pathogens (refs). EDN2 binds to toll-like receptors inside the cell and may serve as an intracellular transporter. EDN2 (nucleotide sequence reference AB100737) has 3 known mutations in cattle, a cDNA of 1247 by and a genomic sequence of 5924 by (Benson et al. 2004). The EDN2 gene is conserved in human, chimpanzee, dog, mouse, rat, and chicken.

The second gene in this region is HIVEP3 (Human immunodeficiency virus type I enhancer-binding protein 3) (SEQ ID NO:2). HIVEP3 is a large gene that covers 54,554 nucleotides (111,560,078-111,614,632 bp) and ends near a SNP (111,623,091 by on chromosome 3) that is associated (P=0.0008) with Map tissue infection. This gene produces proteins that bind specific DNA sequences, including the kappa-B motif (GGGACTTTCC), in the promoters and enhancer regions of several genes involved in immunity, inflammation, and growth. Expression of this gene correlates with the presence of viral antigens, mitogens, cytokines and viruses, including human immunodeficiency virus (HIV).

The LOC521287 (Bos taurus similar to forkhead box O6) (SEQ ID NO:4) gene is conserved in human, mouse, rat, and zebrafish.

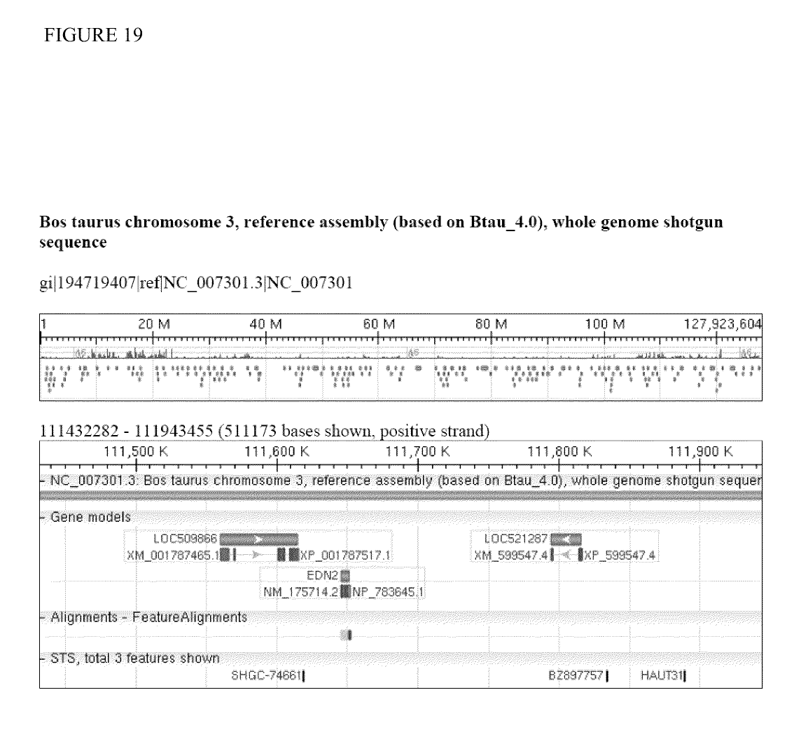

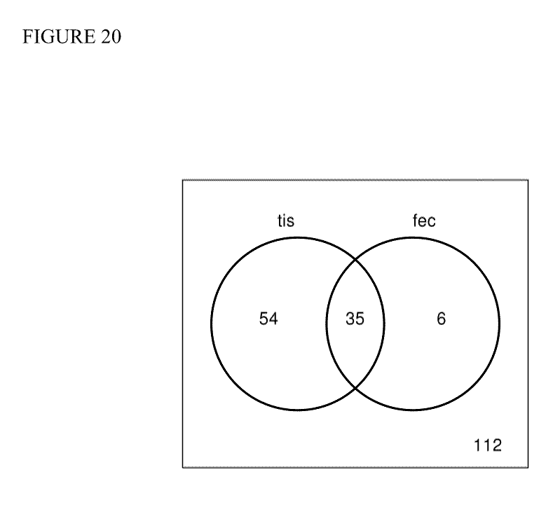

The sequence information for these three genes is shown in Table 5. Additionally, FIG. 19 shows a schematic of the region from chromosome 3 of Bos taurus from position 11432282-111943455 corresponding to NC 007301.3 (based on Btau 4.0), which includes the HIVEP3 (Human immunodeficiency virus type I enhancer-binding protein 3) (SEQ ID NO:2); EDN2 (Endothelin 2) (SEQ ID NO:3); and LOC521287 (Bos taurus similar to forkhead box O6) (SEQ ID NO:4) genes.

TABLE 5
Sequence summary table showing position of the inventive linkage disequilibrium
region, along with the HIVEP3, EDN2 and similar to forkhead box O6
subregions thereof, and including the respective GenBank accession
and version numbers and corresponding SEQ ID NOS: 1-10.

| | Gene sequence: | mRNA sequence: | |
|---|---|---|---|
| | Accession number; | Accession number; | |
| | Version number; | Version number; | Protein sequence: |
| Region or Gene | and chromosome 3 | and chromosome 3 | Accession number and |
| name | position | position | Version number, |
| Linkage | NC_007301.3; | See below | See below |
| disequilibrium | GI:194719407; | | |
| region | 111,560,078 to | | |
| | 111,815,664 | | |
| | (255,586 bp) | | |
| | (SEQ ID NO: 1). | | |
| Or | NC_007301.3; | | |
| Linkage | GI:194719407; | | |
| disequilibrium | 111,586,713 to | | |
| region | 111,821,916 | | |
| | (235,204 bp) | | |
| | (SEQ ID NO: 45) | | |
| HIVEP3 | NC_007301.3; | XM_001787465.1; | XP_001787517.1; |
| Bos taurus similar | GI:194719407; | GI:194665884; | GI:194665885; |
| to human | 111,560,078 to | (mRNA processed | (2,775 amino acids) |
| immunodeficiency | 111,614,644 | 8,328 nt) | (SEQ ID NO: 8) |
| virus type I | (54,567 bp) | (SEQ ID NO: 5) | |
| enhancer binding | (SEQ ID NO: 2) | | |
| protein 3 | | | |
| (LOC509866) | | | |
| EDN2 | NC_007301.3; | NM_175714.2; | NP_783645.1; |
| (Bos taurus | GI:194719407; | GI:31342231 | GI:28372485 |
| Endothelin 2) | 111,645,420 to | (1,249 bp | (177 amino acid |
| | 111,651,344 | processed mRNA) | protein) |
| | (5,925 bp) | (SEQ ID NO: 6) | (SEQ ID NO: 9) |
| | (SEQ ID NO: 3) | | |
| | | See also | See also |
| | | AB100737.1 | BAC55924.1 |
| | | GI:27923033 | GI:27923034 |
| | | (1,247 bp | (177 amino acid |
| | | processed mRNA) | protein) |
| Bos taurus similar | NC_007301.3; | XM_599547.4; | XP_599547.4; |
| to forkhead box | GI:194719407; | GI:194665886 | GI:194665887 |
| O6 | 111,794,197 to | (mRNA processed | (535 amino acids) |
| (LOC521287) | 111,815,664 | 1,608 nt) | (SEQ ID NO: 10) |
| | (21,468 bp) | (SEQ ID NO: 7) | |
| | (SEQ ID NO: 4) | | |

Our genome-wide association study has provided us with strong evidence of association with Map tissue infection that comprised a 235 kb region (SEQ ID NO:45) (and a 255,586 by region (SEQ ID NO:1)) on bovine chromosome 3. According to particular aspects, the EDN2, HIVEP3 and LOC521287 genes have, alone and/or in combination, functions that affect tissue infection of Map in cattle.

To confirm the association study and further characterize the association of Map tissue infection on chromosome 3, 42 additional SNPs were chosen from the NCBI SNP database (http://www.ncbi.nlm.nih.gov/projects/SNP/) to genotype the same animals that were used initially to identify the association of Map tissue infection on chromosome 3. Eighteen of the chosen 42 markers had minor allele frequencies less than 1% in the study animals and were removed from the analysis. Of the SNPs that remained, 17 were in region A in FIG. 2 (mean spacing between SNPs of 1682 bp), 2 in region B (mean spacing of 13,246 bp), 3 in region C (mean spacing of 10,975 bp) and 1 in region D (mean spacing of 69,703 bp). In relation to the genes in this region, exons 4 and 5 of HIVEP3 are located in region A, exons 1 through 4 of EDN2 are located in region B and exon 5 is located in region C. The LOC521287 (Bos taurus similar to forkhead box O6) (SEQ ID NO:4) gene is located to the right in region D. Unfortunately, only two SNPs 3′ to SS86341066 (the SNP with the most evidence for an association with tissue infection) remained after removal of poorly performing or uninformative SNPs. This left a distance of 10 kb and 138 between the last two 3′ SNPs in this region and SS86341066. Twenty-four markers (including a SNP in exon 4 of HIVEP3 and in two non-coding regions of EDN2) were analyzed for an association with tissue infection using chi square analysis.

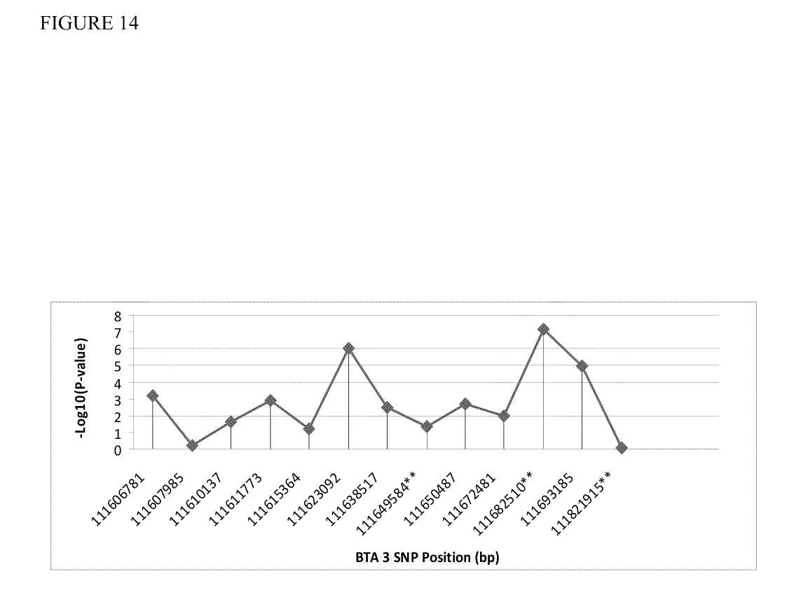

A large region near SS86341066 was found to be associated with Map tissue infection after analysis with the 24 additional SNPs. Thirteen SNPs were associated (P<0.05) with Map tissue infection over the 235 kb region. In a region of 83 kb (111,610,137 to 111,693,185 bp) (SEQ ID NO:47) encompassing SNPs for HIVEP3, EDN2 and SS86341066, ten of ten SNPs were associated with Map tissue infection (FIG. 14) including three SNPs with particularly strong evidence for association (P<2×10⁻⁵).

FIG. 3 shows an association analysis of an 83 kb region of chromosome 3. Location of SNPs (in by with respect to NC_{—}007301.3) are listed on the x axis and the −log 10 (p value) of the association of each SNP with Map tissue infection is listed on the y axis. A −log 10 value of 1.2 represents P=0.05. SNPs with the greatest evidence for association with Map tissue infection include the SNP at 111,623,092 (P=9.4×10⁻⁷), the adjacent 3′ SNP 111,693,185 (P=1.1×10⁻⁵) and the SNP located between HIVEP3 and EDN2 at 111,682,510 by (P=7.5×10⁻⁸). SNPs that are marked with “**” indicate SNPs that were first analyzed with the use of the Illumina bovine SNP50 BeadChip™ and were also included in the fine mapping of additional SNPs in this region.

The association of these SNPs with Map tissue infection is due to linkage disequilibrium. Linkage disequilibrium (LD) refers to correlations among neighboring alleles, reflecting haplotypes descended from single, ancestral chromosomes. Groups of adjacent alleles that have been inherited together can be used to identify genes that cause disease. In the cattle genome, genome-wide LD extends up to an average of 100 kb. This means that within a 100 kb region, it is expected that most of the nucleotides will be inherited together as a block with little recombination or deterioration of LD between the nucleotides over individual generations. Over time, however, LD will slowly deteriorate because of the process of recombination in meiosis. In this process, SNPs that were due to mutations that occurred a long time ago are more likely to have fallen out of LD than SNPs in the same region that are fairly new.

The analysis of SNPs associated with a disease in cattle within a region smaller than 100 kb must considered carefully. Individual SNPs may not provide strong evidence for an association with the disease because that particular SNP may be much older than a nearby “new” SNP that remains in LD with the disease. Because the age of the SNPs are unknown, evaluating the trends of LD within a genomic region of 100 kb or less is particularly helpful. Therefore, it is most important to evaluate the entire region of interest that is associated with the disease rather than solely targeting the DNA immediately adjacent to the SNP with the most evidence for an association. The fine mapping results shown here provide extremely strong evidence of a region and locus that is responsible for differences in Map tissue infection in Holstein cows because an entire block of SNPs are associated with the disease.

Haplotypes were further analyzed for the 83 kb region where the evidence for an association to Map tissue infection was strongest. A haplotype represents the linear arrangement of alleles of the SNPs on one of the two chromosomes in a pair of chromosomes that a cow inherits from her dam or sire. In this haplotype analysis, the complement of alleles on chromosome 3 inherited from the mother (maternal haplotype), and the complement of alleles on chromosome 3 inherited from the father (paternal haplotype) are evaluated separately. The frequencies of the maternal and paternal haplotypes of the Map infected cows are then compared to the haplotype frequencies of the cows that are uninfected.

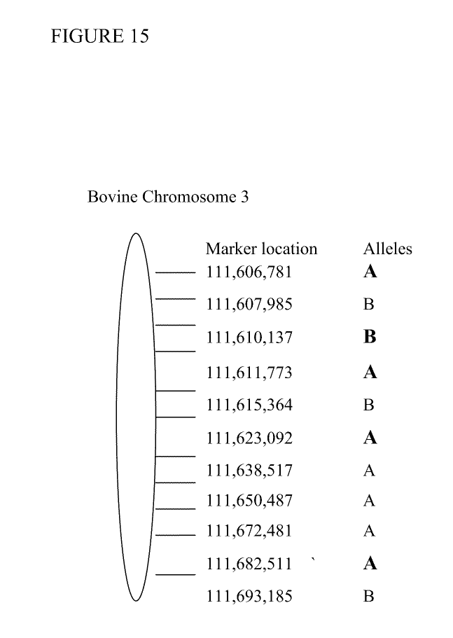

Alleles from 11 SNPs comprised a single haplotype in the 86 kb region (SEQ ID NO:48) (FIG. 15). Twenty-five different combination of alleles for the 11 SNPs were observed and distinct differences were detected between the haplotype frequencies of Map tissue infected animals and uninfected animals (P=2.2×10⁻⁷). The differences in these frequencies demonstrated that certain SNPs in this region were inherited together as a single ancestral block on chromosome 3. This ancestral block also segregated with Map tissue infection. This block represents five SNPs and pinpoints, with very high probability, an underlying sequence that harbors a mutation(s) responsible for resistance to Map.

Alleles of five SNPs (FIG. 15) were different between every Map tissue infected and uninfected cow. Four of these SNPs are adjacent to or span exons in HIVEP3. This data provides strong evidence that this region harbors a mutation that is associated with resistance to Map tissue infection, located in or near exon four or five of HIVEP3.

The data disclosed herein, provide strong evidence of mutations in a 235 kb region (SEQ ID NO:45) (and a 255,586 by region (SEQ ID NO:1) on bovine chromosome 3) that are associated with Map tissue infection, a critical step in the pathogenic process leading to Johne's disease. The 235 kb region was further characterized by increasing the density of SNPs in this area to elucidate a more refined profile of the genome associated with Map tissue infection. This profile confirmed Applicants' initial association results, and identified a haplotype comprising 76 kb (SEQ ID NO:46), from about 111,606,511 to 111,682,511, or from about 111,606,781 to 111,682,511 (comprising 5 SNPs) as particularly preferred region(s) harboring one or more mutation(s) responsible for the resistance of cattle to Map tissue infection. The identification of the linkage disequilibrium regions and mutation(s) disclosed herein provides for compositions and methods for susceptibility, resistance or tolerance to infection by Mycobacteria and Paratuberculosis, and hence to Johne's disease through Map tissue infection.

Nucleic acid sequences encoding mutant nucleic acid and proteins corresponding to HIVEP3 (Human immunodeficiency virus type I enhancer-binding protein 3) (SEQ ID NO:2); EDN2 (Endothelin 2) (SEQ ID NO:3); and LOC521287 (Bos taurus similar to forkhead box O6) (SEQ ID NO:4), and the respective coding transcripts (SEQ ID NOS:5, 6 and 7, respectively), and polypeptides (SEQ ID NOS:8, 9 and 10, respectively) are provide

Nucleic acid sequences comprising one or more nucleotide deletions, insertions or substitutions relative to the wild type nucleic acid sequences are another embodiment of the invention, as are fragments of such mutant nucleic acid molecules. Such mutant nucleic acid sequences (e.g., mutants of genes: HIVEP3 (Human immunodeficiency virus type I enhancer-binding protein 3) (SEQ ID NO:2); EDN2 (Endothelin 2) (SEQ ID NO:3); and LOC521287 (Bos taurus similar to forkhead box O6) (SEQ ID NO:4), and the respective coding transcripts (SEQ ID NOS:5, 6 and 7, respectively), and polypeptides (SEQ ID NOS:8, 9 and 10, respectively) can be generated and/or identified using various known methods, as described further below. Again, such nucleic acid molecules are provided both in endogenous form and in isolated form. In one embodiment, the mutation(s) result in one or more changes (deletions, insertions and/or substitutions) in the amino acid sequence of the encoded protein (i.e. it is not a “silent mutation”). In another embodiment, the mutation(s) in the nucleic acid sequence result in a significantly modulated (up or down), reduced or completely abolished biological activity of the encoded protein relative to the wild type protein.

The nucleic acid molecules may, thus, comprise one or more mutations, such as:

(a) a “missense mutation”, which is a change in the nucleic acid sequence that results in the substitution of an amino acid for another amino acid;

(b) a “nonsense mutation” or “STOP codon mutation”, which is a change in the nucleic acid sequence that results in the introduction of a premature STOP codon and thus the termination of translation (resulting in a truncated protein); plant genes contain the translation stop codons “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation.

(c) an “insertion mutation” of one or more amino acids, due to one or more codons having been added in the coding sequence of the nucleic acid;

(d) a “deletion mutation” of one or more amino acids, due to one or more codons having been deleted in the coding sequence of the nucleic acid;

(e) a “frameshift mutation”, resulting in the nucleic acid sequence being translated in a different frame downstream of the mutation. A frameshift mutation can have various causes, such as the insertion, deletion or duplication of one or more nucleotides.

It is understood that mutations in certain parts of the protein are more likely to result in a reduced function of the mutant HIVEP3, EDN2, or LOC521287 proteins, such as mutations leading to truncated proteins, whereby significant portions of the functional domains are lacking.

Thus in one embodiment, nucleic acid sequences comprising one or more of any of the types of mutations described above are provided. In another embodiment, sequences comprising one or more stop codon (nonsense) mutations, one or more missense mutations and/or one or more frameshift mutations are provided. Any of the above mutant nucleic acid sequences are provided per se (e.g., in isolated form).

### Polypeptide and Nucleic Acid Variants:

Variants of HIVEP3, EDN2, or LOC521287 proteins have utility for aspects of the present invention. Variants can be naturally or non-naturally occurring. Naturally occurring variants (e.g., polymorphisms) are found in various species and comprise amino acid sequences which are substantially identical to the amino acid sequence shown in SEQ ID NOS:8, 9 and 10. Species homologs of the protein can be obtained using subgenomic polynucleotides of the invention, as described below, to make suitable probes or primers for screening cDNA expression libraries from other species, such as human, mice, monkeys, yeast, or bacteria, identifying cDNAs which encode homologs of the protein, and expressing the cDNAs as is known in the art. Orthologs are provided for herein.

Non-naturally occurring variants which retain (or lack) substantially the same biological activities as naturally occurring protein variants are also included here. Preferably, naturally or non-naturally occurring variants have amino acid sequences which are at least 85%, 90%, 95%, 96%, 97%, 98%, 99% or greater than 99% identical to the amino acid sequence shown in SEQ ID NOS:3 or 5. More preferably, the molecules are at least 98%, 99% or greater than 99% identical. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. 2:482-489, 1981.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§.1.821-1.822, abbreviations for amino acid residues are shown in Table 6:

TABLE 6
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Praline |
| K | Lys | Lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

It should be noted that all amino acid residue sequences represented herein by a formula have a left to right orientation in the conventional direction of amino-terminus to carboxyl-terminus. In addition, the phrase “amino acid residue” is defined to include the amino acids listed in the Table of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

Guidance in determining which amino acid residues can be substituted, inserted, or deleted without abolishing biological or immunological activity can be found using computer programs well known in the art, such as DNASTAR software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids. Preferably, amino acid changes in the HIVEP3, EDN2, or LOC521287 proteins polypeptide variants are conservative amino acid changes, i.e., substitutions of similarly charged or uncharged amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting variant. Properties and functions of HIVEP3, EDN2, or LOC521287 protein or polypeptide variants are of the same type as a protein comprising the amino acid sequence encoded by the complements of the respective nucleotide sequence shown above, although the properties and functions of variants can differ in degree.

Variants of the HIVEP3, EDN2, or LOC521287 proteins disclosed herein include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do or do not affect functional activity of the proteins are also variants. Covalent variants can be prepared by linking functionalities to groups which are found in the amino acid chain or at the N- or C-terminal residue, as is known in the art.

A subset of mutants, called muteins, is a group of polypeptides in which neutral amino acids, such as serines, are substituted for cysteine residues which do not participate in disulfide bonds. These mutants may be stable over a broader temperature range than native secreted proteins (see, e.g., Mark et al., U.S. Pat. No. 4,959,314).

It will be recognized in the art that some amino acid sequences of the HIVEP3, EDN2, or LOC521287 proteins of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of ligand binding to cell surface receptors (Ostade et al., Nature 361:266-268, 1993). Thus, the HIVEP3, EDN2, or LOC521287 proteins of the present invention may include one or more amino acid substitutions, deletions or additions, either from natural mutations or human manipulation.

Amino acids in the HIVEP3, EDN2, or LOC521287 proteins of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)).

As indicated, changes in particular aspects are preferably of a minor nature, such as conservative amino acid substitutions that do not significantly affect the folding or activity of the protein. Of course, the number of amino acid substitutions a skilled artisan would make depends on many factors, including those described above. Other embodiments comprise non-conservative substitutions. Generally speaking, the number of substitutions for any given HIVEP3, EDN2, or LOC521287 proteins will not be more than 50, 40, 30, 25, 20, 15, 10, 5 or 3.

All of the compositions, articles, and methods described and claimed herein can be made and executed without undue experimentation in light of the present disclosure. While the invention has been described in terms of preferred embodiments, it will be apparent to those of skill in the art that variations may be applied without departing from the spirit and scope of the invention. All such variations and equivalents apparent to those skilled in the art, whether now existing or later developed, are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method for determining at least one of susceptibility, resistance or tolerance to Mycobacterium avium subspecies paratuberculosis (Map) infection of a subject, comprising:
obtaining a biological sample from a mammalian subject;
determining, using the biological sample, a presence or absence of at least one mutation, or the genotype of at least one single nucleotide polymorphism (SNP) within Map infection linkage disequilibrium region SEQ ID NO:46 that segregates with resistance and/or tolerance to Map tissue infection, or determining a presence or absence of at least one mutation or a genotype of at least one SNP within a Map infection linkage disequilibrium region (SEQ ID NO:1) that is in linkage disequilibrium with the presence or absence of the at least one mutation or with the genotype of the at least one SNP within SEQ ID NO:46; and
determining, based thereon, at least one of susceptibility, resistance or tolerance of the mammalian subject to Mycobacterium avium subspecies paratuberculosis (Map) infection.

2. The method of claim 1, wherein the at least one single nucleotide polymorphism (SNP) within Map infection linkage disequilibrium region SEQ ID NO:46, is at least one selected from the SNP group consisting of, relative to the sequence of accession no. NC_{—}007301.3, SNPs at positions 111,606,781, 111,610,137, 111,611,773, 111, 623,092 and 111,682,511 (ARS-BFGL-NGS-113303) as defined herein.

3. The method of claim 1, wherein the genotypes of at least two single nucleotide polymorphisms (SNPs) within Map infection linkage disequilibrium region SEQ ID NO:46, are determined.

4. The method of claim 1, wherein at least one of resistance or tolerance of the mammalian subject to Mycobacterium avium subspecies paratuberculosis (Map) infection is determined.

5. The method of claim 1, wherein the mammalian subject is bovine.

6. The method of claim 1, further comprising at least one of culling, selecting, or breeding, based on the determining of at least one of susceptibility, resistance or tolerance of the mammalian subject to Mycobacterium avium subspecies paratuberculosis (Map) infection.

7. The method of claim 1, further comprising selective breeding to produce offspring having at least one of susceptibility, resistance or tolerance to Mycobacterium avium subspecies paratuberculosis (Map) infection.

8. A method for determining at least one of susceptibility, resistance or tolerance to Mycobacterium avium subspecies paratuberculosis (Map) infection of a subject, comprising:
obtaining a biological sample from a mammalian subject;
analyzing, using the biological sample, a mammalian genotype to determine if the genotype comprises at least one single nucleotide polymorphism (SNP) associated with at least one of susceptibility, resistance or tolerance to Mycobacterium avium subspecies paratuberculosis (Map), wherein the SNP is at least one selected from the SNP group consisting of, relative to the sequence of accession no. NC_{—}007301.3, SNPs at positions 111,606,781, 111,610,137, 111,611,773, 111, 623,092 and 111,682,511 (ARS-BFGL-NGS-113303) as defined herein; and
determining, based thereon, at least one of susceptibility, resistance or tolerance of the mammalian subject to Mycobacterium avium subspecies paratuberculosis (Map) infection.

9. The method of claim 8, wherein the genotypes of at least two single nucleotide polymorphisms (SNPs) are determined.

10. The method of claim 8, wherein at least one of resistance or tolerance of the mammalian subject to Mycobacterium avium subspecies paratuberculosis (Map) infection is determined.

11. The method of c wherein the mammalian subject is bovine.

12. The method of claim 8, further comprising at least one of culling, selecting, or breeding, based on the determining of at least one of susceptibility, resistance or tolerance of the mammalian subject to Mycobacterium avium subspecies paratuberculosis (Map) infection.

13. The method of claim 8, further comprising selective breeding to produce offspring having at least one of susceptibility, resistance or tolerance to Mycobacterium avium subspecies paratuberculosis (Map) infection.

14. A method for determining at least one of susceptibility, resistance or tolerance to Mycobacterium avium subspecies paratuberculosis (Map) infection of a subject, comprising:
obtaining a biological sample from a mammalian subject;
analyzing, using the biological sample, a mammalian genotype to determine if the genotype comprises at least one single nucleotide polymorphism (SNP) associated with at least one of susceptibility, resistance or tolerance to Mycobacterium avium subspecies paratuberculosis (Map), where the SNP is at least one selected from the SNP group consisting of the SNPs listed in Table 3A and 3B herein; and
determining, based thereon, at least one of susceptibility, resistance or tolerance of the mammalian subject to Mycobacterium avium subspecies paratuberculosis (Map) infection.

15. The method of claim 15, wherein the genotypes of at least two single nucleotide polymorphisms (SNPs) are determined.

16. The method of claim 15, wherein at least one of resistance or tolerance of the mammalian subject to Mycobacterium avium subspecies paratuberculosis (Map) infection is determined.

17. The method of claim wherein the mammalian subject is bovine.

18. The method of claim 8, further comprising at least one of culling, selecting, or breeding, based on the determining of at least one of susceptibility, resistance or tolerance of the mammalian subject to Mycobacterium avium subspecies paratuberculosis (Map) infection.

19. The method of claim 8, further comprising selective breeding to produce offspring having at least one of susceptibility, resistance or tolerance to Mycobacterium avium subspecies paratuberculosis (Map) infection.

20. A method for determining at least one of susceptibility, resistance or tolerance to Mycobacterium avium subspecies paratuberculosis (Map) infection of a subject, comprising:
obtaining a biological sample from a mammalian subject;
determining, using the biological sample, a presence or absence of at least one mutation, or the genotype of at least one single nucleotide polymorphism (SNP) within at least one gene selected from the group consisting of HIVEP3 (Human immunodeficiency virus type I enhancer-binding protein 3) (SEQ ID NO:2); EDN2 (Endothelin 2) (SEQ ID NO:3); and LOC521287 (Bos taurus similar to forkhead box O6) (SEQ ID NO:4) that segregates with resistance and/or tolerance to Map tissue infection, or determining a presence or absence of at least one mutation or a genotype of at least one SNP within a Map infection linkage disequilibrium region (SEQ ID NO:1) that is in linkage disequilibrium with the presence or absence of the at least one mutation or with the genotype of the at least one SNP within within at least one gene selected from the group consisting of HIVEP3 (Human immunodeficiency virus type I enhancer-binding protein 3) (SEQ ID NO:2); EDN2 (Endothelin 2) (SEQ ID NO:3); and LOC521287 (Bos taurus similar to forkhead box O6) (SEQ ID NO:4); and
determining, based thereon, at least one of susceptibility, resistance or tolerance of the mammalian subject to Mycobacterium avium subspecies paratuberculosis (Map) infection.

21. The method of claim 20, wherein the genotypes of at least two single nucleotide polymorphisms (SNPs) are determined.

22. The method of claim 20, wherein at least one of resistance or tolerance of the mammalian subject to Mycobacterium avium subspecies paratuberculosis (Map) infection is determined.

23. The method of claim 20, wherein the mammalian subject is bovine.

24. The method of claim 20, further comprising at least one of culling, selecting, or breeding, based on the determining of at least one of susceptibility, resistance or tolerance of the mammalian subject to Mycobacterium avium subspecies paratuberculosis (Map) infection.

25. The method of claim 20, further comprising selective breeding to produce offspring having at least one of susceptibility, resistance or tolerance to Mycobacterium avium subspecies paratuberculosis (Map) infection.
